(19) United States
(12) Patent Application Publication
(10) Pub. No.: US 2015/0315595 A1
(43) Pub. Date: Nov. 5, 2015

(54) Compositions and Methods for Modulation of ATXN3 Expression

(71) Applicant: Santaris Pharma A/S, Hørsholm (DK)

(72) Inventors: Nathalie Uzcategui, Malmo (SE); Maj Hedtjarn, Copenhagen SV (DK); Jens Bo Rode Hansen, Charlottenlund (DK)

(73) Assignee: SANTARIS PHARMA A/S, Horsholm (DK)

(21) Appl. No.: 14/385,111

(22) PCT Filed: Mar. 12, 2013
(86) PCT No.: PCT/US13/30553

§ 371 Date: Sep. 12, 2014

Related U.S. Application Data
(60) Provisional application No. 61/609,774, filed on Mar. 12, 2012.

(51) Int. Cl. C12N 15/113; A61K 9/00

(52) U.S. Cl. CPC C12N 15/1137; A61K 9/0085; C12N 2310/3231; C12N 2310/341; C12N 2310/3519; C12N 2310/11

(57) ABSTRACT

Disclosed are oligonucleotides which target and hybridize to nucleic acid molecules encoding A TXNJ, leading to reduced expression of ATXN3. Reduction in the expression of aberrant ATXN3 is beneficial for the treatment of certain medical disorders, such as spinocerebellar ataxia 3. In particular embodiments, modulating the expression of an aberrant A TXN3 allele or transcript, for example, restores normal function of, for example, Purkinje cells or spinal cord neurons. The oligonucleotides of the present invention and the methods of using such oligonucleotides to modulate the expression of aberrant or expanded A TXN3 provide a means of improving the survival and morbidity associated with, or even curing, expression of an aberrant A TXN3 allele or transcript such as, for example, spinocerebellar ataxia-type 3 (SCA3).

### RELATED APPLICATION

This application claims priority to, and the benefit of, U.S. Provisional Application No. 61/609,774, filed Mar. 12, 2012. The entire teachings of the above application(s) are incorporated herein by reference.

### FIELD

The present application relates to oligonucleotides and related pharmaceutical compositions that target and hybridize to nucleic acids encoding the protein ataxin-3 (ATXN3) and to methods of using the oligonucleotides to modulate expression of ATXN3 to treat a range of medical disorders, such as spinocerebellar ataxia-type 3 (SCA3).

### BACKGROUND

Spinocerebellar ataxia-type 3 (SCA3), which is also known as Machado-Joseph disease, is an autosomal dominant, progressive neurodegenerative disorder with variable age of onset and severity. SCA3 was originally described in people of Portuguese descent, and in particular from the Azores islands where SCA3 is most prevalent (e.g., the incidence of SCA3 is 1/140 in the small island of Flores) (Sudarsky L., et al., Clin. Neurosci. 1995; 3:17-22). SCA3 was subsequently identified in several other countries and is now considered to be the most common dominantly inherited hereditary ataxia.

Clinically, patients with SCA3 present with progressive gait and limb ataxia, dysarthria and a variable combination of other symptoms including pyramidal signs, dystonia, oculomotor disorders, faciolingual weakness, neuropathy, progressive sensory loss and parkinsonian features. In its more severe forms, SCA3 is characterized by defects in both pyramidal (e.g., motor, somatosensory) and extrapyramidal (e.g., muscle tone) neural functions. Within affected families, this form of ataxia also demonstrates an anticipation effect, which is characterized by an earlier disease onset and more severe symptoms with each new affected generation.

All forms of SCA3 are attributable to an unstable and iterative genetic expansion of a (CAG)ₙ tract in the coding region of ATXN3 on chromosome 14q32.1 that encodes a pathogenic poly-glutamine region or tract in the translated ATXN3 protein (Kawaguchi Y., et al., Nature Genet. 1994; 8: 221-228). The unstable and iterative expansion of the (CAG)ₙ tract in the coding region of ATXN3 (and the pathogenic poly-glutamine tract encoded thereby) causes an increase in protein misfolding, which results in aggregation and formation of nuclear and cytoplasmic inclusions (Paulson H L, et al., 1997, Neuron 19, 333-344). Misfolded protein aggregates are not only a characteristic of SCA3 and Machado-Joseph disease, but are also a common feature of many other neurodegenerative diseases, including Alzheimer's and Parkinson's diseases.

Therapeutic approaches currently available for the treatment of SCA3 are limited to symptomatic treatments, or therapeutic approaches which are based primarily on exercise and diet modification. Efforts made in an attempt to develop therapeutics suitable for the treatment SCA3 have targeted the expanded (CAG)ₙ tract in the coding region of ATXN3. In many instances however, such therapies may not effectively inhibit the expression of a mutated or aberrant allele encoding the pathogenic poly-glutamine tract relative to the functional wild-type allele. For example, International Applications WO2008/018795A1 and WO/2009/099326A1 describe various means of targeting aberrant alleles or transcripts encoding the poly-glutamine expansions by designing oligonucleotides that are complementary to a repeat sequence in both the aberrant and wild-type alleles, but that may preferentially hybridize to the more accessible temporary open loop structure that characterizes the aberrant allele. Similarly, Hu, et al. (Nat. Biotechnol. 2009; 27(5): 478-484) evaluated whether oligomers could discriminate between wild-type and mutant alleles based in part on the structural differences that characterize each of the alleles. Allele-specific silencing of SCA3 has been described by Miller, et al. (Proc. Natl. Acad. Sci. USA 2003; 100(12): 7195-7200) in the context of small interfering RNA-mediated techniques. However, Miller, et al. concluded that a single nucleotide difference between a wild-type and a mutated allele may not be sufficient to confer allele specificity in this context unless specific conditions are met.

Since there are no currently available cures for patients with SCA3, further developments are needed to identify novel therapies which modulate the expression of nucleic acids encoding ATXN3 and which suppress, inhibit, prevent or reduce the expression of ATXN3 that includes the pathogenic poly-glutamine expansion as a means of curing, or at least improving the symptoms of, and the survival and morbidity associated with SCA3 in humans. Particularly needed are novel antisense therapies that are able to effectively target mutated or aberrant alleles encoding the pathogenic poly-glutamine tract on a discriminatory basis relative to the functional wild-type allele.

### SUMMARY

Provided herein are novel oligonucleotides, particularly locked nucleic acid (LNA) antisense oligonucleotides, and therapeutic interventions useful for the treatment of diseases associated with the expression of aberrant, mutated or expanded ATXN3 (e.g., spinocerebellar ataxia-type 3 or Machado-Joseph disease). The inventions disclosed herein relate to the discovery that contacting cells or tissues expressing a mutated or aberrant ATXN3 allele with the oligonucleotides of the present invention modulates the expression of such ATXN3, and in particular modulates the expression of mutated or naturally occurring variants of ATXN3 (e.g., ATXN3 characterized as having a pathogenic, expanded poly-glutamine tract). In particular embodiments, modulating the expression of an aberrant ATXN3 allele or transcript, for example, restores normal function of, for example, Purkinje cells or spinal cord neurons. The oligonucleotides of the present invention and the methods of using such oligonucleotides to modulate the expression of aberrant or expanded ATXN3 provide a means of improving the survival and morbidity associated with, or even curing, expression of an aberrant ATXN3 allele or transcript such as, for example, spinocerebellar ataxia-type 3 (SCA3). In certain embodiments, the oligonucleotides of the present invention, when administered to a subject with SCA3, cause an improvement in or resolution of the symptoms of SCA3 (e.g., improvement in gait and limb ataxia, dysarthria, pyramidal signs, dystonia, oculomotor disorders, faciolingual weakness, neuropathy, progressive sensory loss, lethargy and parkinsonian features).

In one aspect, the inventions disclosed herein relate to oligonucleotides of from about 8 to about 50 nucleotides in length which hybridize to an ATXN3 target sequence (e.g., a mammalian ATXN3 or mRNA sequence encoded thereby). In certain aspects such oligonucleotides hybridize to an ATXN3 target sequence with sufficient stability (e.g., with sufficient hybridization strength and for a sufficient period of time) to inhibit or otherwise modulate expression of an ATXN3 gene product (e.g., an ATXN3 protein characterized as having an expanded pathogenic poly-glutamine tract). Oligonucleotides which are particularly suitable for this purpose and others are described herein.

In one aspect, the present invention provides oligonucleotides of from about 8 to about 50 nucleotides in length (e.g., from about 8 to 30, 8 to 20, 12 to 18, or 14 to 16 nucleotides in length) which comprise a contiguous nucleotide sequence (a first region) of from about 8 to about 30 nucleotides (e.g., about 8, 9, 10, 11, 12, 13, 14, 15, 16, 17, 18, 19, 20, 21, 22, 23, 24, 25, 26, 27, 28, 29, or 30 nucleotides in length) having at least 80% identity (e.g., at least about 85%, 90%, 95%, 96%, 97%, 98%, 99% or 100% identity) with a region corresponding to the reverse complement of the coding region of a mammalian ATXN3 gene or the complement of mRNA encoding ATXN3. For example, the oligonucleotides of the present invention may comprise a contiguous nucleotide sequence which is at least 80% complementary to a portion of a nucleic acid sequence encoding ATXN3 (e.g., at least 80% complementary to a portion of a nucleic acid sequence encoding ATXN3 DNA, pre-mRNA or mRNA). The oligonucleotides disclosed herein may comprise a nucleic acid sequence that is complementary to a region of a mutated ATXN3 gene or to the corresponding mRNA encoded thereby. Similarly, the oligonucleotides disclosed herein may comprise a sequence that is complementary to the gene product of an ATXN3 gene (e.g., mRNA encoded by the ATXN3 gene) or a polymorph or naturally-occurring variant thereof that encodes a mutation such as a the region encoding the pathogenic poly-glutamine expansion (CAG)ₙ and which, for example comprises a 0987C single nucleotide polymorphism, as is encoded for example by SEQ ID NO: 4, or naturally-occurring variants thereof (e.g., the transcript variants encoded by NM_{—}004993.5 or single nucleotide polymorphisms such as SNP ID rs12895357). In particular, the oligonucleotides described herein may be at least 80% complementary (e.g., at least about 85%, 90%, 95%, 96%, 97%, 98%, 99% or more complementary) to a nucleic acid sequence encoding a mutated region of an ATXN3 gene or mRNA, such as a region encoding the poly-glutamine expansion mutation and the regions immediately upstream and/or downstream of the region encoding the pathogenic poly-glutamine expansion region (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250 or more nucleotides upstream and/or downstream from the location of the poly-glutamine expansion region).

In another aspect, the invention provides oligonucleotides comprising about 8 to about 20 nucleotides, wherein the oligonucleotides hybridize to at least an 8-nucleobase portion of a nucleic acid encoding ATXN3 (e.g., ATXN3 mRNA). For example, in some embodiments the oligonucleotides of the present invention hybridize to the nucleic acids (i.e., mRNA) encoding the poly-glutamine expansion region, or to a region immediately surrounding and/or adjacent to the nucleic acids encoding the poly-glutamine expansion region (e.g., the G→C single nucleotide polymorphism which is located one nucleotide downstream or 3′ of the pathogenic (CAG)ₙ expansion and that is referred to herein as the “G987C” SNP or mutation). In some embodiments, the oligonucleotides are complementary to a region of a single stranded nucleic acid molecule encoding ATXN3, such as, for example a region of a nucleic acid molecule having the sequence of a portion of SEQ ID NO: 4 or naturally occurring variants thereof (e.g., SNP ID rs12895357).

In some embodiments, the claimed oligonucleotides comprise a sequence which is complementary to a DNA sequence encoding ATXN3 mRNA or a portion thereof, or alternatively the claimed oligonucleotides hybridize to an RNA sequence (e.g., pre-mRNA or mRNA) or portion thereof encoded thereby. When brought into contact with targeted cells or tissues (e.g., the neurons or other tissues of the central nervous system of a patient affected by or afflicted with SCA3) the oligonucleotides disclosed herein can reduce the expression of ATXN3 (and in particular reduce expression of mutated or aberrant ATXN3), and thereby restoring neuronal function. For example, the oligonucleotides of the present invention can target, and in certain embodiments hybridize to the nucleic acids (e.g., mRNA) encoding mutated or aberrant ATXN3, such as, for example, the mRNA comprising or encoded by SEQ ID NO: 13, SEQ ID NO: 14, SEQ ID NO: 15, SEQ ID NO: 16, SEQ ID NO: 17 and/or SEQ ID NO: 18, or a particular portion or region of any of the foregoing (e.g., the region encoding the pathogenic poly-glutamine expansion) and thereby modulate the expression of ATXN3, such that expression is reduced and/or inhibited by at least about 10%, 20%, 25%, 35%, 40%, 50%, or preferably at least 60%, 65%, 70%, 75%, 85%, 90%, 95%, 99% or 100%.

In yet another aspect, the invention provides compositions comprising oligonucleotides such as those described herein. In some embodiments, the compositions can include a pharmaceutical composition comprising one or more oligonucleotides described herein together with one or more pharmaceutically acceptable excipients, adjuvants, or other molecules to facilitate or improve the delivery or stability of the composition. In some embodiments, the inventions provide for a conjugate comprising one or more oligonucleotides described herein and at least one non-nucleotide or non-polynucleotide moiety attached thereto, for example, covalently or non-covalently attached to said oligonucleotide. Also disclosed herein are oligonucleotides and conjugates and pharmaceutical compositions comprising the same for use as a medicament, such as for the treatment of diseases associated with the expression of aberrant ATXN3 (e.g., ataxias such SCA3 and Machado-Joseph disease), and methods of treating such diseases by administering the oligonucleotides, conjugates and/or pharmaceutical compositions described herein to a mammalian subject, for example, a human subject such as a paediatric human subject (before or after birth) or an adult human subject.

In another aspect, the inventions provide for the use of an oligonucleotide or a conjugate thereof for the manufacture of a medicament for the treatment of SCA3. Also contemplated by the present inventions is the use of the oligonucleotides described herein (e.g., an oligonucleotide that hybridizes to a region of SEQ ID NO: 4 or SNP ID rs12895357 comprising a G987C single nucleotide polymorphism) as a medicament.

Similarly, provided herein are uses of the oligonucleotides described herein (e.g., oligonucleotides that hybridize to mRNA encoding or adjacent to the ATXN3 poly-glutamine expansion tract) in or for the treatment of diseases such as SCA3. The invention also provides for methods of treating diseases or conditions associated with the expression of nucleic acids encoding mutated or aberrant ATXN3, such as SCA3 or Machado-Joseph disease, the methods comprising the steps of administering an effective amount of an oligonucleotide, a conjugate and/or a pharmaceutical composition according to the invention, to a subject suffering from, likely to suffer from or otherwise affected by or afflicted with SCA3 (e.g., such as a human paediatric or adult patient suffering from or susceptible to SCA3). In some embodiments, the disease, disorder or condition associated with the expression of aberrant ATXN3 relates to the over-expression of ATXN3, and in particular the over-expression of the mutated or expanded ATXN3 (e.g., ATXN3 comprising an unstable and/or iterative genetic pathogenic expansion of a (CAG)ₙ tract, where “n” equals or is greater than 52). In some embodiments, the oligonucleotides, conjugates and pharmaceutical compositions described herein preferentially modulate the expression of an ATXN3 mutant, polymorph or naturally occurring variant, such as for example an ATXN3 mutant, polymorph or naturally occurring variant which comprises a pathogenic poly-glutamine expansion (CAG)ₙ, (e.g., as is encoded by SEQ ID NO: 4 or SNP ID rs12895357). Such preferential modulation of the expression of an ATXN3 mutant, polymorph or naturally occurring variant by the oligonucleotides of the present invention may be partial or absolute in nature relative to the expression of wild-type ATXN3 (e.g., as is encoded by SEQ ID NO: 1). For example, when administered to a patient with (heterozygous) SCA3, the oligonucleotides of the present invention may target both mRNA encoding the wild-type ATXN3 allele and mRNA encoding a mutated or expanded ATXN3 allele, however such oligonucleotides may modulate the expression of each target to a varying extent, such that, for example, the expression of the expanded ATXN3 allele is modulated to a greater extent than is the expression of the wild-type ATXN3 allele. The oligonucleotides of the present invention may, for example, target and reduce the expression of a mutated ATXN3 variant or polymorph that comprises a G987C transition substitution (e.g., an ATXN3 variant or polymorph comprising a sequences encoded by SEQ ID NOS: 4, 5 or 6) by a factor of 2, 4, 8, 10, 15, 25, 50, 75, 100, 200 or more; while the same oligonucleotide respectively reduces the expression of a wild-type ATXN3 (e.g., as encoded by SEQ ID NO: 1) by a factor of 1, 2, 4, 5, 10, 15, 25, 50, 75, 100 or more. Similarly, the oligonucleotides of the present invention may, for example, target and reduce the expression of a mutated ATXN3 polymorph or variant that comprises a pathogenic poly-glutamine region (e.g., as is encoded by SEQ ID NO: 4 or SNP ID rs12895357) by about 1%, 2.5%, 5%, 10%, 20%, 35%, 40%, 50%, 60%, 75%, 80%, 85%, 90%, 95%, 97.5%, 99% or more; while the same oligonucleotide reduces the expression of a wild-type ATXN3 (e.g., as encoded by SEQ ID NO: 1) by about 1%, 2.5%, 5%, 10%, 20%, 35%, 40%, 50%, 60%, 75%, 80% or 90%. Also disclosed herein are oligonucleotides which target and/or hybridize (e.g., specifically hybridize) to nucleic acids encoding mutated or expanded ATXN3 on a discriminatory basis relative to nucleic acids that encode a functional or wild-type ATXN3. For example, in a patient with Machado-Joseph disease the oligonucleotides of the invention may target and reduce the expression of a mutated or expanded ATXN3 allele by about 1%, 2.5%, 5%, 10%, 20%, 35%, 40%, 50%, 60%, 75%, 80%, 90%, 95%, 97.5%, 99%, or more relative to the expression of a functional or wild-type ATXN3 allele. Alternatively, the oligonucleotides of the present invention may increase the expression of a wild-type ATXN3 gene product or mRNA (e.g., in a paediatric patient affected by SCA3) while reducing and/or inhibiting the expression of a mutated ATXN3 gene product or mRNA. In some embodiments the oligonucleotides, conjugates and pharmaceutical compositions disclosed herein reduce or otherwise inhibit expression of mutated ATXN3 (e.g., by preferentially targeting and hybridizing to nucleic acids (e.g., mRNA) which encode the ATXN3 pathogenic (CAG)ₙ expansion and/or the G987C single nucleotide polymorphism in an allele of a patient with SCA3), while not affecting or minimally affecting the expression of ATXN3 which does not encode the mutation.

In some embodiments, the oligonucleotides disclosed herein hybridize (e.g., specifically hybridize) to the gene product of ATXN3 (i.e., mRNA), for example, the mRNA gene product encoded by a mutated ATXN3 polymorph or variant which comprises a pathogenic (CAG)ₙ mutation or expansion (e.g., as is encoded by SEQ ID NO: 4 or SNP ID rs12895357). In other embodiments, the oligonucleotides hybridize to the gene products (e.g., mRNA) of the nucleic acids encoding a mutated or expanded ATXN3 polymorph or variant, where the nucleotides encoding such ATXN3 polymorph comprise a pathogenic (CAG)ₙ mutation or region (e.g., a pathogenic (CAG)ₙ, where “n” equals 81). In other embodiments, the oligonucleotides of the present invention may specifically hybridize to the gene products of the nucleic acids (i.e., mRNA) encoding a mutated ATXN3 polymorph or variant (e.g., as is encoded by SEQ ID NO: 4 or SNP ID rs12895357), while the same oligonucleotide does not specifically hybridize to the gene products of the nucleic acids (i.e., mRNA) encoding the wild-type ATXN3 (e.g., as is encoded by SEQ ID NO: 1). Such preferential or discriminatory hybridization of the oligonucleotides to the nucleic acids encoding an expanded or mutated ATXN3 polymorph, can modulate the expression of the expanded or mutated gene product while the expression of the wild-type ATXN3 gene product is preserved or otherwise remains unchanged. For example, the oligonucleotides of the present invention may target and preferentially hybridize to mRNA encoded by nucleic acid comprising SEQ ID NOS: 15-20 (or a fragment thereof), such that the expression of the protein encoded by such mRNA is reduced and/or inhibited by at least about 10%, 20%, 25%, 35%, 40%, 50%, or preferably at least 60%, 65%, 70%, 75%, 85%, or most preferably at least 90%, 95%, 99% or 100%.

In some embodiments, the oligonucleotides of the present invention hybridize to the nucleic acids (e.g., mRNA) encoding human ATXN3 (e.g., the ATXN3 mRNA encoded by Accession Number NM_{—}004993, inclusive of any variants and polymorphs thereof). For example, the oligonucleotides of the present invention may target and hybridize to the human ATXN3 mRNA (e.g., as is encoded by SEQ ID NO: 1 and/or SEQ ID NO: 4). Also contemplated are oligonucleotides that preferentially hybridize to one or more target sequences, wherein such target sequences comprise ATXN3 mRNA (e.g., target sequences that comprise one or more of SEQ ID NO: 13, SEQ ID NO: 14, SEQ ID NO: 15, SEQ ID NO: 16, SEQ ID NO: 17 or SEQ ID NO: 18). The oligonucleotides of the present invention may preferably hybridize to human ATXN3 mRNA which comprises a pathogenic (CAG)ₙ mutation, expansion or to a fragment thereof (e.g., the ATXN3 mRNA encoded by Accession Number NM_{—}004993), and thus modulate the expression of the targeted human ATXN3. Alternatively, the same oligonucleotide may specifically hybridize to the nucleic acids encoding an ATXN3 polymorph which encodes a pathogenic (CAG)ₙ mutation or expansion, but may not hybridize to the nucleic acids encoding the wild-type of the human species which lacks or does not otherwise encode that pathogenic (CAG)ₙ mutation or expansion under the same or similar stringency conditions.

Also provided are methods of inhibiting the expression of nucleic acids encoding mutated, expanded or aberrant ATXN3, and in particular methods of inhibiting the production (e.g., transcription or translation) of the gene products of such nucleic acids encoding mutated, expanded or aberrant ATXN3 gene (e.g., mRNA encoding expanded ATXN3), in a cell (e.g., a Purkinje cell or neuron) which is expressing a mutated or expanded ATXN3. In some embodiments, the method comprises administering an oligonucleotide, conjugate or pharmaceutical composition according to the invention to a patient, or otherwise contacting a cell or tissue with such oligonucleotide, conjugate or pharmaceutical composition so as to inhibit the expression of ATXN3 (e.g., ATXN3 comprising a pathogenic poly-glutamine expansion) in such patient or cell.

Also disclosed are oligonucleotides of from about 8 to 50 monomers, which comprise a first region of about 8 to 50 contiguous monomers (e.g., nucleotides), wherein the sequence of such first region is at least 80% identical (e.g., at least 85%, at least 90%, at least 95%, at least 99% identical) to one or more selected target sequences (e.g., a target sequence comprising mRNA encoding mutated or expanded ATXN3). In some embodiments, the selected target sequences may comprise a region of nucleic acids encoding mammalian ATXN3 (e.g., mRNA) or a fragment thereof. Further provided are locked antisense oligonucleotides, for example, 8 to 50, 12 to 30 or 12 to 20 nucleotides in length. For example, in some embodiments the oligonucleotides comprise one or more locked nucleic acid (LNA) residues or monomeric units (e.g., SEQ ID NO: 19, SEQ ID NO: 20, SEQ ID NO: 21, SEQ ID NO: 22 or SEQ ID NO: 23). Where the oligonucleotides of the present invention comprise two or more LNA monomeric units (e.g., two or more 13-D-oxy-LNA monomeric units), such LNA monomeric units may be located consecutively relative to each other, or alternatively such LNA monomeric units may be located non-consecutively relative to each other. In certain embodiments, the oligonucleotides disclosed herein are gapmers. For example, disclosed herein are antisense oligonucleotides comprising SEQ ID NO: 12, wherein the oligonucleotides modulate expression of ATXN3, and wherein the oligonucleotides comprise at least one locked nucleic acid at one or more nucleotides selected from the group consisting of: wherein said oligonucleotide modulates expression of ATXN3, and wherein said oligonucleotide comprises at least one nucleotide analogue at one or more positions selected from the group consisting of: (i) the adenine nucleotide at one or more of positions 1 and 3 is an oxy-LNA; (ii) the guanine nucleotide at position 10 is an oxy-LNA; (iii) the cytosine nucleotide at one or more of positions 9 and 11 is an oxy-LNA; and (iv) the thymine nucleotide at position 2 is an oxy-LNA.

Optionally, such locked antisense oligonucleotides may comprise one or more sugar substitutions, such as for example, a 2′-O-methoxyethyl sugar substitution. Also provided herein are conjugates which comprise one or more of the oligonucleotides according to the invention, wherein such oligonucleotides comprise at least one non-nucleotide or non-polynucleotide moiety which is covalently attached to the oligonucleotide of the invention.

Also provided are pharmaceutical compositions which comprise one or more of the oligonucleotides or the conjugates according to the invention, and a pharmaceutically acceptable diluent, carrier, salt, solvent or adjuvant. Also provided are pharmaceutical compositions which comprise one or more of the oligonucleotides of the invention. Such pharmaceutical compositions may be administered, for example, parenterally by injection or infusion directly to the target site of action or may be administered by inhalation, peritoneally, topically, orally or intrathecally.

Further provided are methods of down-regulating the expression of an allele or nucleic acids encoding mutated or aberrant ATXN3 (e.g., down-regulating expression of expanded ATXN3 at the mRNA level), and in particular down-regulating the expression of mutant or naturally-occurring variants of ATXN3, in cells or tissues. Such methods comprise contacting the cells or tissues, in vitro or in vivo, with an effective amount of one or more of the oligonucleotides, conjugates or compositions of the invention. In some embodiments, the oligonucleotides and compositions of the present invention are capable of down-regulating the expression of a mutated, expanded or aberrant ATXN3 allele in a mammal (e.g., in a human patient suffering from or otherwise affected by SCA3) while not modulating or otherwise minimally affecting the expression of a normally functioning or wild-type allele.

Also disclosed are methods of treating an animal (e.g., a non-human animal or a human) suspected of having, or susceptible to, a disease or condition, associated with the expression, or over-expression of expanded or aberrant ATXN3 by administering to the animal a therapeutically or prophylactically effective amount of one or more of the oligonucleotides, conjugates or pharmaceutical compositions described herein. Furthermore, provided herein are methods of using oligonucleotides to inhibit the expression of mutated or aberrant ATXN3 (e.g., mutated or naturally-occurring variants of ATXN3).

Also provided are methods of treating conditions associated with the expression of aberrant ATXN3 (e.g., ataxias such as SCA3 and Machado-Joseph disease) and methods of restoring cellular (e.g., neuronal) function. Such methods comprise delivering to, or contacting affected cells that express an aberrant allele of ATXN (e.g., neurons or Purkinje cells), with one or more of the oligonucleotides of the present invention. The conditions under which the claimed method introduces the oligonucleotides to the neuronal cells (e.g., transfection or gymnotic delivery) are sufficient to reduce expression of an aberrant ATXN3 allele in the affected cells, and thereby restore normal cellular function. In some embodiments, such methods preferentially reduce the expression of an expanded ATXN3 polymorph or naturally-occurring variant which comprises a G987C single nucleotide polymorphism. The invention provides for methods of treating a disease such as SCA3 and Machado-Joseph disease, the method comprising administering an effective amount of one or more oligonucleotides, conjugates, or pharmaceutical compositions thereof to a patient in need thereof (e.g., a human paediatric patient affected by SCA3). The invention provides for methods of inhibiting (e.g., by down-regulating) the expression of mutated or aberrant ATXN3 in a cell or a tissue, the method comprising the step of contacting the cell or tissue with an effective amount of one or more oligonucleotides disclosed herein or conjugates or pharmaceutical compositions thereof, to thereby down-regulate the expression of the mutated or aberrant ATXN3 allele (e.g., at the mRNA level).

The above discussed, and many other features and attendant advantages of the present invention will become better understood by reference to the following detailed description of the invention when taken in conjunction with the accompanying examples.

### BRIEF DESCRIPTION OF THE FIGURES

FIGS. 1A and 1B illustrate the flag-tagged mutant (MUT) reporter construct and the wild-type (WT) endogenous expression reporter construct levels in HEK-293 cells having undergone 48 hours of gymnotic treatment with oligonucleotides complementary to portions of the region of the expanded ATXN3 allele that include the G987C single nucleotide polymorphism one nucleotide from the pathogenic (CAG)ₙ expansion of the ATXN3 mRNA. The HEK-293 cells were stably transfected with a pFLAG-ATX3Q81-FL-FFLuciferase reporter construct that comprised the coding region of the mutated ATXN3 transcript having the G987C SNP and 81 (CAG) repeats fused to a firefly luciferase transcript and the oligonucleotides were subsequently introduced into the transfected cells by gymnosis at media concentrations of 1 μM, 5 μM and 25 μM. The cells were then harvested after 48 hours, and the percentage of mRNA expression determined using the relevant qPCR assay. The results are expressed as a percentage of the mock-treated samples and are reported as the average of three independent studies per oligonucleotide. The depicted error bars indicate the standard deviation. As illustrated in both FIGS. 1A and 1B, the 20 oligonucleotides demonstrated a robust knock-down of the MUT reporter construct with a marked dose response relative to the endogenous WT reporter.

FIGS. 2A and 2B illustrate the expression of the expanded ATXN3 transcript (MUT) and the endogenous ATXN3 (WT) mRNA in the ATXN3-Q81 transfected HEK-293 cells following gymnotic delivery of twelve selected oligonucleotides. The results were normalized to the endogenous GAPDH levels and expressed as a percent of the mock treated samples. The oligonucleotides were introduced to the cells by gymnosis using media having final concentrations of 0.3 μM, 1 μM, 3 μM, 9 μM, 27 μM and 81 μM. The cells were then harvested 48 hours after gymnotic delivery of the oligonucleotides, and the mRNA of the mutated ATXN3 transcript (reporter construct) and the endogenous ATXN3 transcript were extracted and analyzed by qPCR. The results are reported as the average of three independent studies per oligonucleotide and the error bars indicate the standard deviation. As illustrated in FIGS. 2A and 2B, all twelve oligonucleotides evaluated were found to produce a dose-dependent knock-down of the MUT reporter construct in the concentration ranges evaluated relative to the WT reporter construct.

FIG. 3 illustrates the half maximal inhibitory concentration (IC₅₀) curves for the five oligonucleotides that were selected as leads and designed to be complementary to portions of the region of the expanded ATXN3 allele that includes the G987C single nucleotide polymorphism located one nucleotide from the pathogenic (CAG)ₙ expansion of the ATXN3 mRNA. HEK-293 cells were stably transfected with a pFLAG-ATX3Q81-FL-FFLuciferase reporter construct that comprised the coding region of the mutated ATXN3 transcript having the G987C SNP and 81 (CAG) repeats fused to a firefly luciferase transcript and the oligonucleotides were subsequently introduced into the transfected cells by gymnosis using media having final concentrations of 0.3 μM, 3 μM, 9 μM, 27 μM and 81 μM. The cells were then harvested 48 hours after gymnotic delivery of the oligonucleotides, and the mRNA of the mutated ATXN3 transcript (reporter construct) and the endogenous ATXN3 transcript were extracted and analyzed by qPCR. The plotted data points represent the average reporter signal of three independent experiments and the error bars represent the standard deviation. The grey curve (♦) represents the fitted response curve of the wild-type (WT) reporter and the black curve (▴) represents the fitted response curve of the expanded or mutant (MUT) reporter. The corresponding IC₅₀ value for each curve is indicated on each graph and the actual values for the five selected lead oligonucleotides are also reported in Table 2.

FIG. 4 illustrates the stability of each of twelve oligonucleotides complementary to portions of the region of the expanded ATXN3 allele that includes the G987C single nucleotide polymorphism located one nucleotide from the pathogenic (CAG)ₙ expansion of the ATXN3 mRNA. Each of the oligonucleotides was incubated in cerebrospinal fluid with added brain tissue for 120 hours at 37° C. Samples were taken at 0, 24, 48, 96 and 120 hours and analyzed by SDS-PAGE. The plasma stability of each of the twelve oligonucleotides was found to be well within the expected ranges, and in particular all of the oligonucleotides were found to have an overall half-life greater than 96 hours and most of the oligonucleotides did not produce any appreciable degradation products.

FIGS. 5A and 5B illustrate the results of a 16-day in vivo tolerance study in which oligonucleotides complementary to portions of the region including and surrounding the nucleotides encoding the G987C single nucleotide polymorphism were administered to mice on days 0, 3, 7, 10 and 14. The mice were sacrificed and evaluated at day 16. The control administered was a saline control. The results are reported as the average of five independent studies per oligonucleotide and the error bars indicate the standard deviation. As shown in FIGS. 5A and 5B, the five selected oligonucleotides (SH06, SH10, SH13, SH16 and SH20, which correspond to SEQ ID NOS: 19, 20, 21, 22 and 23, respectively) resulted in negligible elevations of the liver enzymes alanine aminotranferease (ALT) and aspartate aminotransferase (AST) relative to the saline control.

### DETAILED DESCRIPTION

The oligonucleotides described herein provide specific therapeutic tools capable of modulating the expression of ATXN3. In some embodiments, the short (e.g., usually about less than 50, 40, 30, 20, 18, 17, 16, 15, 14, 13, 12, 11, 10, 9, 8 or less nucleotides in length) single-stranded synthetic oligonucleotides described herein have a base sequence complementary to the ATXN3 RNA target sequence (e.g., pre-mRNA or mRNA) and form a hybrid duplex by hydrogen bonded base pairing. For example, in some embodiments the oligonucleotides of the present invention may target or be complementary to nucleic acids encoding ATXN3 (e.g., mRNA encoding ATXN3) or a fragment thereof (e.g., SEQ ID NOS: 5 or 6) and thereby modulate the expression of ATXN3. In other embodiments, the oligonucleotides of the present invention (e.g., locked nucleic acid gapmers) may generally work by a cleavage mode of action or sterically blocking enzymes involved in processing pre-mRNA or translation of mRNA. This hybridization can be expected to prevent expression, (i.e., translation of the target mRNA code into its protein product) and thus preclude subsequent effects of the protein product. Accordingly, the oligonucleotides and methods described herein can be used to ameliorate or treat one or more conditions (e.g., diseases or syndromes) associated with the expression of aberrant ATXN3, for example, ataxias such spinocerebellar ataxia-type 3 (SCA3).

SCA3, which is also known as Machado-Joseph disease, is an autosomal dominant, progressive neurodegenerative disorder with variable age of onset and severity. SCA3 is caused by a pathogenic (CAG)ₙ trinucleotide expansion in the nucleic acids that encode ataxin-3, which results in the expansion of a poly-glutamine domain or tract in the ATXN3 protein. SCA3 was originally described in people of Portuguese descent, and in particular from the Azores islands where SCA3 and Machado-Joseph disease are most prevalent (e.g., the incidence of SCA3 is 1/140 in the small island of Flores). (Sudarsky L., et al., Clin. Neurosci. 1995; 3:17-22.) SCA3 was subsequently identified in several other countries and is now considered to be the most common dominantly inherited hereditary ataxia.

Clinically, SCA3 and Machado-Joseph disease present with progressive gait and limb ataxia, dysarthria and a variable combination of other symptoms including pyramidal signs, dystonia, lethargy, oculomotor disorders, faciolingual weakness, neuropathy, progressive sensory loss and parkinsonian features. In its more severe forms, SCA3 is characterized by defects in both pyramidal (e.g., motor, somatosensory), extrapyramidal (e.g., muscle tone) and neural functions. Within affected families, this form of ataxia also demonstrates an anticipation effect, which is characterized by an earlier disease onset and more severe symptoms with each new affected generation.

As with the clinical features, the underlying degenerative changes in SCA3 vary to some degree. Among the central nervous system regions that undergo neuronal loss and reactive astrogliosis are select telencephalic, cerebellar and brainstem nuclei, the anterior horn of the spinal cord, Clarke's column and the dorsal root ganglia. (Dürr A, et al., Ann Neurol 1996; 39: 490-9.) The molecular pathogenesis of SCA3 remains speculative since the normal function of ATXN3 is poorly understood, however all forms of SCA3 are attributable to an unstable and iterative genetic expansion of a (CAG)ₙ tract in the coding region of ATXN3 on chromosome 14q32.1. (Kawaguchi Y., et al., Nature Genet. 1994; 8: 221-228.) ATXN3 is a polyubiquitin-binding protein whose physiological function has been linked to ubiquitin-mediated proteolysis. (Doss-Pepe E W, Mol, Cell. Biol. 2003; 23:6469-6483.) The presence of the expanded (CAG)ₙ mutation in the nucleic acids encoding ATXN3 results in a long poly-glutamine chain at the C-terminus region of the ATXN3 protein and that is referred to herein as the “poly-glutamine expansion” or the “poly-glutamine tract”. (Dtirr A, et al., Ann Neurol 1996; 39: 490-9.) As used herein, the term “expanded” refers to the presence of the poly-glutamine expansion (e.g., an expanded allele having (CAG)ₙ wherein n is greater that 52). The poly-glutamine expansion increases protein misfolding, which results in aggregation and formation of nuclear and cytoplasmic inclusions. (Paulson H L, et al., 1997, Neuron 19, 333-344.) The poly-glutamine expansion is also associated with the formation of harmful ubiquinated nuclear aggregates and inclusions in Purkinje cells and spinal cord neurons. Misfolded protein aggregates are not only a characteristic of SCA3, but are also a common feature of many other neurodegenerative diseases, including Alzheimer's and Parkinson's diseases.

Because of the dominant inheritance pattern of SCA3, and because loss of ATXN3 function does not confer the same disease phenotype as the poly-glutamine expansion, the expanded allele acquires a dominant toxicity in the affected neuron. (Schmitt et al., Biochem Biophys Res Commun 2007; 362(3):734-9.) Toxicity may arise from saturation of the ubiquinin/proteasome machinery, or other regulatory defects arising from the accumulation of unfolded or misfolded proteins, or from defective protein degradation.

Individuals who are unaffected by SCA3 normally demonstrate between about 10-40 glutamine repeat lengths in the ATXN3 protein (e.g., (CAG)ₙ where “n” equals about 10-40), compared to patients with SCA3 who may demonstrate between about 55-84 or more expanded glutamine repeat lengths, which is generally referred to herein as the “pathogenic (CAG)ₙ expansion” or the “pathogenic expansion” (e.g., (CAG)ₙ where n=about 55-84 or more). (See, Paulson, H L, Intranuclear inclusions of expanded polyglutamine protein in spinocerebellar ataxia type 3/Machado Joseph disease. BIOS Scientific Publishers, Oxford, United Kingdom; Kawaguchi Y., et al. Nat. Genet. 1994; 8: 221-228.) The expansion of the poly-glutamine tract confers a toxic gain-of-function on the mutated ATXN3 protein, which leads to the formation of neuronal intranuclear inclusions. (Schmidt T, et al., Brain Pathol. 1998; 8:669-679). The length of the poly-glutamine tract inversely correlates with age at onset of the SCA3. (Netravathi M., et al., J. Neurol Sci. (2009) 277 1-2:83-6).

The loss of function of the wild-type ATXN3 allele has also been shown to play a role in ubiquitin-mediated proteolysis and such loss of function may be deleterious. (Doss-Pepe E W, Mol. Cell. Biol. 2003; 23:6469-6483.) Accordingly, a strategy in which the oligomeric compounds of the present invention solely target the pathogenic (CAG)ₙ expansion may not be beneficial. Rather, in certain embodiments a strategic and discriminatory targeting of the mutated (e.g., disease-causing) ATXN3 allele may be preferred.

A strategic targeting of the mutated ATXN3 allele which is based on the presence of a single nucleotide polymorphism (SNP) has been proposed to ensure discrimination between the wild-type and mutant ATXN3 alleles. (Miller V M, et al. Proc Natl Acad Sci. 2003; 100:7195-7200.) That SNP, which is located one nucleotide downstream or 3′ of the pathogenic (CAG)ₙ expansion of the mutated ATXN3 allele, is in linkage disequilibrium with the disease-causing poly-glutamine expansion and typically segregates with the diseased allele. (Stevanin G, et al. Am J Hum Genet. 1995; 57:1247-1250; and Gaspar C, et al. Hum Genet. 1996; 98:620-624.) In most SCA3 patients, the wild-type ATXN3 allele has a G at position 987, whereas the expanded/mutant ATXN3 allele has a C at position 987. (Gaspar C, et al. Am J Hum Genet. 2001; 68:523-528). All mutant ATX3 alleles encoding the poly-glutamine expansion have a C at position 987, while approximately 50% of wild-type alleles have a G at this position.

Reduced expression of the wild-type ATXN3 allele does not lead to haploinsufficiency, and accordingly a reduced expression of the wild-type ATXN3 allele is not expected to be detrimental. The presence of the G-to-C SNP or mutation (referred to herein as the “G987C” SNP or mutation) therefore provides an opportunity to use the oligomeric compounds of the present invention to discriminatorily target the expanded or mutated ATXN3 allele, while preserving function of the wild-type ATXN3 allele.

Accordingly, in one embodiment the present disclosures relates to an allele-specific targeting or knockdown of the mutant or expanded allele encoding ATXN3 which is responsible for the development of SCA3. A special consideration in this regard is that, given the extreme instability of (CAG)ₙ repeat, duplication of the (CAG)ₙ tract is common. As previously discussed, normal alleles have been shown to contain between about 13 and 36 CAG repeats; however, in certain instances the normal allele may contain as many as 47 CAG repeats. SCA3 disease pathology occurs in expanded alleles with more extreme duplications, for example in excess of 52 CAG repeats. (See, Kawaguchi Y., et al. Nat. Genet. 1994; 8: 221-228.)

The oligonucleotides, pharmaceutical compositions and methods described herein can be used to ameliorate or treat ataxias such as SCA3, for example, by modulating the expression or function of one or more aberrant nucleic acid molecules (e.g., expanded ATXN3).

### Oligonucleotides

In some embodiments the oligonucleotides described herein target nucleic acids encoding aberrant ATXN3 (e.g., mRNA encoding ATXN3 as provided in SEQ ID NO: 4 and/or fragments thereof as provided in SEQ ID NO: 5 and SEQ ID NO: 6) and naturally occurring variants of such nucleic acids, and thereby modulate expression of ATXN3. As used herein, the term “oligonucleotide” refers to a molecule formed by the covalent linkage of two or more nucleotides. The term oligonucleotide generally includes oligonucleotide analogues, oligonucleotide mimetics and chimeric combinations of these. In the context of the present invention, a single nucleotide unit may also be referred to as a monomer or unit. In some embodiments, the terms “nucleoside”, “nucleotide”, “unit” and “monomer” are used interchangeably. It will be recognized that when referring to a sequence of nucleotides or monomers, what is referred to is the sequence of bases, such as, for example A, T (or U), G, or C.

In some embodiments, the oligonucleotides disclosed herein are useful for modulating the expression of nucleic acid molecules (e.g., modulating the expression of aberrant ATXN3) via an antisense mechanism of action. This modulation may be accomplished, for example, by providing oligonucleotides which are complementary to and/or hybridize to one or more target nucleic acid molecules, such as mRNA (e.g., SEQ ID NO: 4 or SNP ID rs12895357). In some embodiments, the oligonucleotides of the present invention are complementary to a specific region of a target nucleic acid (e.g., the region of the nucleic acid encoding ATXN3 that is adjacent to or surrounding the G987C transition substitution located immediately downstream (3′) of the pathogenic (CAG)ₙ expansion). In some embodiments, the oligonucleotides of the present invention are capable of hybridizing (e.g., specifically hybridizing in physiological conditions) to a specific region of a target nucleic acid (e.g., the region of ATXN3 mRNA encoding the G987C SNP or transition substitution).

As used herein, the phrase “target nucleic acid” is intended to encompass DNA and RNA (including pre-mRNA and mRNA or portions thereof) transcribed from such DNA, and also cDNA derived from such RNA. For example, in some embodiments, the phrase “target nucleic acid” is used to refer to nucleic acids encoding ATXN3 (e.g., mRNA), or in particular nucleic acids encoding mutated or aberrant ATXN3. As used herein, the term “gene product” refers to any biochemical materials resulting from expression of a gene or nucleic acid (e.g., DNA or RNA) and include, but are not limited to mRNA, RNA and/or proteins. For example, in some embodiments, when used with respect to the ATXN3 gene the phrase gene product refers to mRNA encoded by ATXN3. In certain embodiments, the target nucleic acid comprises a nucleic acid sequence selected from the group consisting of SEQ ID NO: 1, SEQ ID NO: 2, SEQ ID NO: 3, SEQ ID NO: 4, SEQ ID NO: 13, SEQ ID NO: 14, SEQ ID NO: 15, SEQ ID NO: 16, SEQ ID NO: 17 and SEQ ID NO: 18. In other embodiments, the oligonucleotides disclosed herein are complementary to and/or hybridize to a nucleic acid sequence comprising one or more of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 14, SEQ ID NO: 15, SEQ ID NO: 16, SEQ ID NO: 17 and/or SEQ ID NO: 18.

In some embodiments, the oligonucleotide compounds of the present invention are complementary to one or more target nucleic acids (e.g., mRNA encoding ATXN3) and interfere with the normal function of the targeted nucleic acid (e.g., by an antisense mechanism of action). This interference with or modulation of the function of a target nucleic acid by the oligonucleotides of the present invention which specifically hybridize to it is generally referred to as “antisense”. The functions of DNA to be interfered with may include replication and transcription. The functions of RNA to be interfered with may include functions such as, for example, translocation of the RNA to the site of protein translation, translation of protein from the RNA, splicing of the RNA to yield one or more mRNA species, and catalytic activity which may be engaged in or facilitated by the RNA. In some embodiments, the overall effect of interference with a target nucleic acid function is modulation of the expression of the product of such target nucleic acid.

As the phrases are used herein, “antisense compound” and “antisense oligonucleotide” refer to an oligonucleotide that is at least partially complementary (e.g., 100%, about 99%, 98%, 97.5%, 95%, 90%, 85%, 80%, 75%, 70%, 65%, 60%, 55%, or 50% complementary) to the region of a nucleic acid molecule, and in particular a target nucleic acid such as the mRNA encoding an aberrant or mutated protein or enzyme. In some embodiments, the antisense compound or antisense oligonucleotide is capable of hybridizing to a target nucleic acid, thereby modulating its expression.

The oligonucleotides of the present invention consist of or comprise a contiguous nucleotide sequence of from about 8 to 50 nucleotides in length, such as for example 8 to 30 nucleotides in length. In various embodiments, the compounds of the invention do not comprise RNA units or monomers, but rather, for example, comprise DNA units or monomers and/or in some instances LNA units or monomers. It is preferred that the compound according to the invention is a linear molecule or is synthesized as a linear molecule. In some embodiments the oligonucleotide is a single stranded molecule, and preferably does not comprise short regions of, for example, at least 3, 4 or 5 contiguous nucleotides, which are complementary to equivalent regions within the same oligonucleotide (i.e., duplexes). In this regard, the oligonucleotide is not essentially double stranded.

### The Target Sequences

In certain embodiments, the oligonucleotides described herein are capable of modulating, or in some embodiments down-regulating (e.g. reducing or eliminating) the expression of the ATXN3 (e.g., down-regulating translation of nucleic acids encoding aberrant or mutated ATXN3 at the mRNA level). In this regards, the oligonucleotides of the invention can affect the inhibition of ATXN3, typically in a mammalian cell such as a human cell (e.g., an A549 cell, a HeLa cell, a Purkinje cell or a neuronal cell). In some embodiments, the oligonucleotides of the invention hybridize to the target nucleic acid (e.g., mRNA encoding an expanded, mutated or aberrant ATXN3 mRNA) and affect inhibition or reduction of expression of at least about 10%-100% compared to the normal expression level (e.g., such as the expression level in the absence of the oligonucleotide or conjugate). For example, the oligonucleotides disclosed herein may affect at least about a 5%, 10%, 20%, 25%, 30%, 40%, 50%, 60%, 70%, 80%, 90%, 95%, 97.5%, 98%, 99% or 100% reduction or inhibition of the expression of ATXN3 compared to the normal expression level of ATXN3 seen an individual carrying an ATXN3 mutant allele. In some embodiments, such modulation is evident upon exposing a targeted cell or tissue to a concentration of about 0.04 nM-25 nM (e.g., a concentration of about 0.8 nM-20 nM) of the compound of the invention. In the same or a different embodiment, the inhibition of expression of the target nucleic acid (e.g., mRNA encoding mutated ATXN3) is less than 100% (e.g., such as less than about 98% inhibition, less than about 95% inhibition, less than about 90% inhibition, less than about 80% inhibition or less than about 70% inhibition). In some embodiments, the oligonucleotides disclosed herein are capable of modulating expression of ATXN3 at the mRNA level (e.g., by targeting and hybridizing to mRNA encoding mutated or aberrant ATXN3). Modulation of expression (e.g., at the mRNA level) can be determined by measuring protein levels or concentrations (e.g., by SDS-PAGE followed by Western blotting using suitable antibodies raised against the target protein). Alternatively, modulation of expression (e.g., at the mRNA level) can be determined by measuring levels or concentrations of mRNA, (e.g., by Northern blotting or quantitative RT-PCR). When measuring expression via the evaluation of mRNA levels or concentrations, the degree of down-regulation when using an appropriate dosage or concentration of an oligonucleotide (e.g., about 0.04 nM-25 nM, or about 0.8 nM-20 nM), can be greater than about 10%, from about 10-20%, greater than about 20%, greater than about 25%, or greater than about 30% relative to the normal levels or concentrations observed in the absence of the oligonucleotide, conjugate or composition of the invention.

In the context of the present invention, the terms “modulating” and “modulation” can mean one or more of an increase (e.g., stimulation or upregulation) in the expression of a gene or gene product (e.g., ATXN3 mRNA), a decrease (e.g., downregulation or inhibition) in the expression of a gene or gene product (e.g., ATXN3 mRNA), and a change in the relative expression between two or more gene products (e.g., a reduction in the expression of mutant ATXN3 relative to the expression of wild-type ATXN3). In some contexts described herein, downregulation and inhibition are the preferred forms of modulation, in particular as it relates to modulating the expression of mutated or expanded ATXN3 (e.g., ATXN3 comprising a pathogenic poly-glutamine tract). In some contexts described herein, the term “expression” means the process by which information from a gene or nucleic acid (e.g., DNA) is used in the synthesis of gene products (e.g., mRNA, RNA and/or proteins) and includes, but is not limited to, one or more of the steps of replication, transcription and translation. The steps of expression which may be modulated by the oligonucleotides of the present invention may include, for example, transcription, splicing, translation and post-translational modification of a protein.

As it relates to targeting, modulation and expression, the term “ATXN3” broadly can refer to the ataxin-3 gene or its gene product (e.g., pre-mRNA, mature mRNA, cDNA, or protein) and can include both mutated and wild-type forms, isoforms and variants thereof (e.g., the nucleic acids encoding human ATXN3 and coding for ATXN3 protein). The italicized term, “ATXN3” as used herein typically refers to the ataxin-3 gene. The term “wild-type” as it describes ATXN3, refers to the most frequently observed ATXN3 allele, nucleotide sequence, amino acid sequence, or phenotype in a subject or population. For example, relative to a mutated ATXN3 allele characterized by the presence of a nucleic acid encoding that expanded poly-glutamine tract, the term “wild-type” refers to the remaining allele that does not comprise such mutation. The terms “mutant” and “mutated” as they describe ATXN3 refers to an altered allele, nucleotide sequence, amino acid sequence, or phenotype in a subject or population that comprises, for example, one or more transition and transversion point mutations that result in the replacement of a single base nucleotide with another nucleotide of the genetic material (e.g., DNA or RNA). An example of a mutation is the single nucleotide polymorphism in ATXN3 which is a G-to-C transition substitution immediately 3′ to the pathogenic (CAG)ₙ expansion and is located at position 987 of NM_{—}004993 (inclusive of any variants and polymorphs thereof which comprise the same G-to-C transition substitution), and which is referred to herein as the “0987C” mutation or SNP. The G987C mutation is in linkage disequilibrium with the disease causing poly-glutamine expansion and accordingly in most patients with SCA3 the G987C mutation segregates with the allele encoding the pathogenic poly-glutamine expansion. The SNP ID for the G987C mutation is SNP ID rs12895357. The term “mutant” and “mutated” are also meant to include transition and transversion point mutations that result in the replacement of a single base nucleotide with another nucleotide of the genetic material, DNA or RNA. Such a mutation is exemplified by the G987C transition substitution adjacent to a pathogenic (CAG)ₙ expansion, which in most patients with SCA3 presents as a C at position 987 instead of a G (as in normal patients).

As used herein, the terms “expansion” or “expanded” as they describe or qualify ATXN3 or the nucleic acids encoding ATXN3, refer to a region of iterative genetic duplications in the genetic code, and in particular the region comprising the (CAG)ₙ tri-nucleotide repeats (e.g., nucleic acids encoding ATXN that comprise a region (CAG)ₙ, where “n” is greater than about 52) that encodes the pathogenic poly-glutamine expansion. An example of an expansion is the iterative genetic duplication of an unstable (CAG)ₙ repeat in the nucleic acid encoding ATXN3, which encodes and results in a pathogenic poly-glutamine expansion in the translated ATXN3 protein product.

As it specifically relates to ATXN3, the phrase “modulating the expression” means a stimulation, upregulation, downregulation, and/or inhibition of the gene products of the ATXN3 gene (e.g., the gene products of the wild-type and/or mutated ATXN3). For example, the oligonucleotides of the present invention that target the nucleic acids (e.g., mRNA) encoding aberrant ATXN3 and specifically hybridize to such nucleic acids (e.g., mRNA encoding ATXN3) can modulate the expression ATXN3. The oligonucleotides described herein can modulate the expression of both wild-type and mutated ATXN3 in patients with Machado-Joseph disease or SCA3. Alternatively, in preferred embodiments, the oligonucleotides described herein can preferentially downregulate or inhibit the expression of mutant or expanded ATXN3 (e.g., the oligonucleotides described herein may modulate the expression of the mutant ATXN3 characterized by the presence of pathogenic (CAG)ₙ tri-nucleotide repeats).

In some embodiments, the oligonucleotides of the present invention are capable of targeting specific nucleic acids. Targeting in the context of the antisense oligonucleotides described herein to a particular nucleic acid can be a multi-step process. The process usually begins with the identification of a nucleic acid sequence whose function is to be modulated. This may be, for example, a nucleic acid (e.g., mRNA) whose expression is associated with a particular disorder or disease state (e.g., SCA3). In some embodiments, the target nucleic acid (e.g., mRNA) encodes ATXN3. In some embodiments the oligonucleotides of the present invention are capable of causing an allele-specific modulation of a target nucleic acid (e.g., by selectively targeting an allele encoding a pathogenic poly-glutamine tract to thereby modulate expression of an expanded allele on a discriminatory basis relative to the functional wild-type allele). For example, in certain embodiments the target nucleic acid may comprise a region or fragment of the nucleic acid gene encoding the G987C transition substitution and/or the (CAG)ₙ expansion tract of ATXN3. Alternatively, in some embodiments the target nucleic acid encodes a particular region of ATXN3 (or the corresponding mRNA) which comprises the G987C mutation and/or the (CAG)ₙ expansion tract of ATXN3. The targeting process also can include a determination of a site or sites within the target gene for the antisense interaction to occur such that one or more desired effects will result. The one or more desired effects can include, for example, modulation of expression of a gene product (e.g., wild-type and/or mutant mRNA or protein), selective binding (e.g., increased binding affinity) for the target site relative to other sites on the same gene or mRNA or on other genes or mRNAs, sufficient or enhanced delivery to the target, and minimal or no unwanted side effects. In some embodiments, a preferred targeted nucleic acid or mRNA site encodes the G987C SNP and/or the (CAG)ₙ expansion tract of ATXN3 and/or the adjacent regions.

The poly-glutamine expansion represents the most common mutation responsible for the development of spinocerebellar ataxia 3. As described above, the term “poly-glutamine expansion” refers to an iterative and pathogenic repeat of glutamine residues which is encoded by the (CAG)ₙ tri-nucleotide (referred to herein as the “pathogenic (CAG)ₙ expansion” where n≧about 52) and that may be present in nucleic acids encoding ATXN3. The poly-glutamine expansion encoded by (CAG)ₙ, where n≧about 52 has been associated with SCA3, while n≦about 47 may be more common and less likely to cause SCA3. The poly-glutamine expansion also refers to a region immediately surrounding the pathogenic (CAG)ₙ tri-nucleotide repeat, for example, the region measuring 2, 5, 10, 12, 20, 30, 50, 60, 75, 80, or 100 codons upstream and downstream from such poly-glutamine expansion (e.g., the G987C mutation). The poly-glutamine expansion confers a toxic gain-of-function on ATXN3, which leads to the formation of neuronal intranuclear inclusions, and represents the most common mutation responsible for the development of SCA3. In the context of the present invention, the term “single-nucleotide polymorphism” or “SNP” refers to a variation in the nucleotide sequence occurring when a single nucleotide differs between members of a species or between paired chromosomes in an individual, for example, the G987C SNP located at the 3′ end of the pathogenic (CAG)ₙ expansion.

The poly-glutamine expansion confers a disease-causing gain-of-function on ATXN3, and accordingly the antisense oligomeric compounds that selectively downregulate the mutated or expanded ATXN3 allele are predicted to improve or restore normal ATXN3 function of the remaining (wild-type) allele. Specifically, in patients with SCA3 the oligomeric compounds of the present invention target and hybridize to the nucleic acids (i.e., mRNA) encoding a mutated ATXN3 allele (e.g., a nucleic acid comprising SEQ ID NO: 4 or SNP ID rs12895357) on a discriminatory basis, such that expression of the mutated ATXN3 allele is downregulated or inhibited, while the same compound does not target or hybridize to the wild-type allele (e.g., SEQ ID NO: 1) or does so to a lesser extent, thus preserving the function of the remaining wild-type allele. The oligonucleotides described herein may be delivered to one or more of an animal, a mammal, a human, or a cell. Targeted cell types may, in some embodiments, include neuronal cells, brain cells, Purkinje cells, HeLa cells, HEK-293 or A549 cells. In certain embodiments, the oligonucleotide concentration used (e.g., in HEK-293 or A549 cells) may be about 0.025 nM, 0.03 nM, 0.05 nM, 0.1 nM, 0.25 nM, 0.27 nM, 0.3 nM, 0.5 nM, 0.81 nM, 0.9 nM, 1 nM, 2.5 nM, 5 nM, 40 nM, 100 nM, 200 nM, 250 nM or more. The oligonucleotide concentration used may, in some embodiments be 25 nM (e.g., in Purkinje cells). In the absence of a transfection reagent (e.g., using gymnotic delivery) a media characterized as having an oligonucleotide concentration between about 1 μM-25 μM (e.g., such as about 5 μM) may be used to downregulate the target gene.

The oligonucleotide concentration used may, in some embodiments be 0.1 nM-1 nM (e.g., in neuronal cells). In certain embodiments, the oligonucleotides disclosed herein may be periodically administered to a subject (e.g., administered intravenously or subcutaneously to a human on a daily, weekly, monthly, quarterly, semi-annually or annual basis) at a dose of about 0.2 to about 20 mg/kg (e.g., administered in daily or weekly doses of at least about 0.2 mg/kg, 0.25 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 6.0 mg/kg, 7.5 mg/kg, 8.0 mg/kg, 10 mg/kg, 12.5 mg/kg, 15 mg/kg or 20 mg/kg). It should be noted that in some embodiments the determination of the appropriate concentration of oligonucleotide used to treat the cell may be performed in an in vitro cell assay using a transfection reagent (e.g., LIPOFECTIN).

In some embodiments, the oligonucleotides described herein are potent inhibitors of ATXN3 (i.e., are capable of modulating the expression of ATXN3 in a cell or tissue upon exposing such cell or tissue to a relatively low concentration of the oligonucleotide). In some embodiments, the oligonucleotides are capable of reducing or otherwise inhibiting the expression of ATXN3 (e.g., of mutated ATXN3) at relatively low concentrations of such oligonucleotide. For example, in some embodiments an oligonucleotide may inhibit expression of ATXN3 by a cell at a relatively low concentration (e.g., an IC₅₀ of less than about 5 nM as determined by a transfection assay, or an IC₅₀ of less than about 4 nM, such as less than 2 nM). As used herein, the term “IC₅₀” refers to the concentration of an oligonucleotide that is sufficient to inhibit an objective parameter (e.g., ATXN3 protein expression) by about fifty percent. In certain embodiments, the antisense oligonucleotides disclosed herein are characterized as selectively inhibiting the expression of mutant ATXN3 protein relative to the expression of wild-type ATXN3 protein. Accordingly, an oligonucleotide may be characterized as inhibiting the expression of mutant ATXN3 protein at a lower concentration (e.g., about two-fold lower) relative to the concentration required to inhibit expression of a wild-type ATXN3 protein. For example, the antisense oligonucleotides may demonstrate at least a two-fold difference in the IC₅₀ for the mutant and wild-type ATXN3 proteins (e.g., at least about a 2-, 2,5-, 3-, 4-5-, 6-, 7-, 8-, 9- or 10-fold difference in the IC₅₀ required to inhibit expression of the ATXN3 mutant protein relative to the normal or wild-type protein in a mammal with SCA3).

The invention therefore provides methods of modulating (e.g., downregulating or inhibiting) the expression of mRNA encoding an expanded or aberrant ATXN3 protein and/or ATXN, and in particular ATXN3 mRNA comprising or encoding the poly-glutamine expansion, in a cell expressing such expanded or mutated ATXN3 protein and/or mRNA (e.g., a Purkinje cell expressing the mutant ATXN3 protein and/or mRNA). Such methods comprise administering the oligonucleotide or conjugate according to the invention to a cell (or otherwise contacting such cell with such oligonucleotide or conjugate) to downregulate or inhibit the expression of ATXN3 protein and/or mRNA in said cell. In some embodiments, the cell can be an in vitro or in vivo mammalian cell, such as a human cell. For example, an oligonucleotide of the present invention that targets nucleic acids encoding an expanded or mutated ATXN3 and specifically hybridizes to the gene product thereof and thereby modulate the expression of the expanded or mutated ATXN3. The oligonucleotides of the present invention may modulate the expression of wild-type and/or mutated ATXN3 alleles in patients with SCA3. The administration to the patient (e.g., human or mammalian), subject (e.g., human or mammalian), and/or cell (e.g., human or mammalian) may occur in vivo, ex vivo, or in vitro. For example, in some embodiments, the oligonucleotide in a pharmaceutically acceptable formulation and/or in a pharmaceutically acceptable carrier or delivery vehicle may be administered directly into the patient's or subject's body, by methods described herein. Alternatively, in some embodiments, the oligonucleotide may be administered to cells after they are removed and before they are returned to the patient's or subject's body. In some embodiments, the cells may be maintained under culture conditions after they are removed and before they are returned to the patient's or subject's body.

The phrase “target nucleic acid”, as used herein refers to the nucleic acids (e.g., mRNA) encoding mammalian ATXN3, and in particular refers to the nucleic acids (e.g., mRNA) encoding mutated or aberrant ATXN3. For example, disclosed herein are target nucleic acids which encode ATXN3 that comprise the pathogenic poly-glutamine expansion (e.g., such as is encoded by SEQ ID NO: 4). Suitable target nucleic acids include nucleic acids encoding ATXN3 or naturally occurring variants thereof, and RNA nucleic acids derived therefrom (e.g., mRNA target sequences comprising or corresponding to SEQ ID NOS: 15-20), preferably mRNA, such as pre-mRNA, although preferably mature mRNA. In some embodiments (e.g., when used in a research or diagnostic context) the “target nucleic acid” may be a cDNA or a synthetic oligonucleotide derived from the above DNA or RNA nucleic acid targets. The oligonucleotides according to the invention are capable of hybridizing to the target nucleic acid or to the gene product of such target nucleic acid. It will be recognized that in some embodiments the target nucleic acid sequence is a cDNA sequences and as such, corresponds to the mature mRNA target sequence, although uracil may be replaced with thymidine in the cDNA sequences.

The term “naturally occurring variant thereof” refers to variants of the ATXN3 polypeptide or nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the ATXN3 encoding genomic DNA that is found at the chromosome by chromosomal translocation or duplication, and the RNA, such as mRNA derived therefrom. For example, naturally occurring variants of ATXN3 may include the G987C mutant, as is encoded for example by SEQ ID NO: 4, or the naturally occurring variants thereof (e.g., SNP ID rs12895357). Naturally occurring variants may also include variants derived from alternative splicing of the ATXN3 mRNA. When referenced to a specific polypeptide sequence the term also includes naturally occurring forms of the protein which may therefore be processed, for example, by co- or post-translational modifications (e.g., signal peptide cleavage, proteolytic cleavage, glycosylation, etc.)

### Sequences

In some embodiments the oligonucleotides comprise or consist of a contiguous nucleotide sequence which corresponds to the reverse complement of a nucleotide sequence of SEQ ID NO: 1 or SEQ ID NO: 4, or a fragment of SEQ ID NO: 1 or SEQ ID NO: 4. Thus, the oligonucleotide can comprise or consist of a sequence selected from the group consisting of SEQ ID NOS: 9, 10, 11, 12, 13 or 14, wherein said oligonucleotide (or contiguous nucleotide portion thereof) may optionally have one, two, or three mismatches against the selected target sequence. In some embodiments, the oligonucleotides may comprise or consist of a contiguous nucleotide sequence which corresponds to the reverse complement of a nucleotide sequence encoding the ATXN3 sequence region that includes the G987C SNP and nucleotides surrounding such SNP. For example, in some embodiments the oligonucleotides may comprise the sequences identified in Table 1 (i.e., SEQ ID NO: 7, SEQ ID NO: 8, SEQ ID NO: 9, SEQ ID NO: 10, SEQ ID NO: 11 or SEQ ID NO: 12). The oligonucleotides may be complementary to a region of a nucleic acid (e.g., mRNA) encoding ATXN3 that includes the G987C mutation (e.g., a region which is about 1, 2, 3, 4, 5, 6, 7, 8, 9, 10, 11, 12, 13, 14, 15, 16, 17, 18, 19, 20 or more nucleotides upstream and/or downstream from the G987C mutation), such as the target sequences identified in Table 1. For example, in some embodiments the oligonucleotides may be complementary to the mRNA target sequences identified in Table 1 (e.g., SEQ ID NO: 13, SEQ ID NO: 14, SEQ ID NO: 15, SEQ ID NO: 16, SEQ ID NO: 17 or SEQ ID NO: 18). In some embodiments, such complementary oligonucleotides are capable of hybridizing (e.g., specifically hybridizing) to the gene product of ATXN3 (i.e., ATXN3 mRNA), and in particular the gene product of ATXN3 comprising the G987C SNP.

TABLE 1

| | | mRNA Target | |
|---|---|---|---|
| Oligonucleotide | | Sequence | |
| SEQ ID NO | Oligonucleotide Sequence | Identifier | mRNA Target Sequence |
| SEQ ID NO: 7 | 5′ - ATAGGTCCCGCTGCT - 3′ | SEQ ID NO: 13 | 5′ - AGCAGCGGGACCUAU - 3′ |
| SEQ ID NO: 8 | 5′ - TGATAGGTCCCGCTGC - 3′ | SEQ ID NO: 14 | 5′ - GCAGCGGGACCUAUCA - 3′ |
| SEQ ID NO: 9 | 5′ - CTGATAGGTCCCGCTC - 3′ | SEQ ID NO: 15 | 5′ - CAGCGGGACCUAUCAG - 3′ |
| SEQ ID NO: 10 | 5′ - CTGATAGGTCCCGCT - 3′ | SEQ ID NO: 16 | 5′ - AGCGGGACCUAUCAG - 3′ |
| SEQ ID NO: 11 | 5′ - CTGATAGGTCCCGC - 3′ | SEQ ID NO: 17 | 5′ - GCACCUAUCAG - 3′ |
| SEQ ID NO: 12 | 5′ - ATAGGTCCCGC - 3′ | SEQ ID NO: 18 | 5′ - GCGGGACCUAU - 3′ |

The oligonucleotide may comprise or consist of a contiguous nucleotide sequence which is fully complementary (perfectly complementary) to the equivalent region of a nucleic acid which encodes a mammalian ATXN3 (e.g., SEQ ID NO: 1, SEQ ID NO: 4 or a fragment thereof). Thus, the oligonucleotide can comprise or consist of an antisense nucleotide sequence capable of hybridizing to the nucleic acids encoding ATXN3 (i.e., ATXN3 mRNA).

However, in some embodiments, the oligonucleotide may tolerate 1, 2, 3 or 4 (or more) mismatches, when hybridizing to the target sequence and still sufficiently bind to the target to show the desired effect (e.g., downregulation of the target mRNA). Mismatches may, for example, be compensated by increased length of the oligonucleotide sequence and/or an increased number of nucleotide analogues, such as locked nucleic acids (LNA), present within the nucleotide sequence. In some embodiments, the contiguous nucleotide sequence comprises no more than 3 mismatches (e.g., no more than 1 or no more than 2 mismatches) when hybridizing to a target sequence, such as to the corresponding region of a nucleic acid which encodes a mammalian ATXN3 mRNA. In some embodiments, the contiguous nucleotide sequence comprises no more than a single mismatch when hybridizing to the target sequence, such as the corresponding region of a nucleic acid which encodes a mammalian ATXN3 mRNA.

The nucleotide sequence of the oligonucleotides of the invention or the contiguous nucleotide sequence is preferably at least 80% complementary to a sequence selected from the group consisting of SEQ ID NOS: 15, 16, 17, 18, 19 or 20, such as at least 85%, at least 90%, at least 91%, at least 92%, at least 93%, at least 94%, at least 95%, at least 96%, at least 97%, at least 98%, at least 99%, such as at least 100% complementary.

The nucleotide sequence of the oligonucleotides of the invention or the contiguous nucleotide sequence is preferably at least 80% homologous to the reverse complement of a corresponding sequence present in SEQ ID NO: 4, such as at least 85%, at least 90%, at least 91%, at least 92%, at least 93%, at least 94%, at least 95%, at least 96% homologous, at least 97% homologous, at least 98% homologous, at least 99% homologous, such as 100% homologous (identical).

The nucleotide sequence of the oligonucleotides of the invention or the contiguous nucleotide sequence is preferably at least 80% complementary to a sub-sequence present in SEQ ID NO: 4, such as at least 85%, at least 90%, at least 91%, at least 92%, at least 93%, at least 94%, at least 95%, at least 96% complementary, at least 97% complementary, at least 98% complementary, at least 99% complementary, such as 100% complementary (perfectly complementary).

In some embodiments the oligonucleotide (or contiguous nucleotide portion thereof) is selected from, or comprises, one of the sequences selected from the group consisting of SEQ ID NOS: 9, 10, 11, 12, 13 or 14, or a sub-sequence of at least about 6-10 contiguous nucleotides thereof. In some embodiments, said oligonucleotide (or contiguous nucleotide portion thereof) may optionally comprise one, two, or three mismatches when compared to the sequence.

In some embodiments the sub-sequence may consist of 8, 9, 10, 11, 12, 13, 14, 15, 16, 17, 18, 19, 20, 21, 22, 23, 24, 25, 26, 27, 28, or 29 contiguous nucleotides, such as from about 12-22, such as from about 12-18 nucleotides. Suitably, in some embodiments, the sub-sequence is of the same length as the contiguous nucleotide sequence of the oligonucleotide of the invention.

In some embodiments the oligonucleotide according to the invention consists or comprises of a nucleotide sequence according to SEQ ID NOS: 9, 10, 11, 12, 13 or 14, or a sub-sequence of thereof.

In some embodiments the oligonucleotide according to the invention consists of or comprises a nucleotide sequence according to SEQ ID NO: 7 or a sub-sequence of thereof.

In some embodiments the oligonucleotide according to the invention consists of or comprises a nucleotide sequence according to SEQ ID NO: 8 or a sub-sequence of thereof.

In some embodiments the oligonucleotide according to the invention consists of or comprises a nucleotide sequence according to SEQ ID NO: 9 or a sub-sequence of thereof.

In some embodiments the oligonucleotide according to the invention consists of or comprises a nucleotide sequence according to SEQ ID NO: 10 or a sub-sequence of thereof.

In some embodiments the oligonucleotide according to the invention consists of or comprises a nucleotide sequence according to SEQ ID NO: 11 or a sub-sequence of thereof.

In some embodiments the oligonucleotide according to the invention consists of or comprises a nucleotide sequence according to SEQ ID NO: 12 or a sub-sequence of thereof.

In determining the degree of complementarity between the oligonucleotides of the invention (or regions thereof) and the target region of a nucleic acid (e.g., mRNA encoding mammalian ATXN3 protein) the degree of complementarity (or homology or identity) is expressed as the percentage identity (or percentage homology) between the sequence of the oligonucleotide (or region thereof) and the sequence of the target region (or the reverse complement of the target region) that best aligns therewith. The percentage is calculated by counting the number of aligned bases that are identical between the 2 sequences, dividing by the total number of contiguous monomers in the oligonucleotide, and multiplying by 100. In such a comparison, if gaps exist, it is preferable that such gaps are merely mismatches rather than areas where the number of monomers within the gap differs between the oligonucleotide of the invention and the target region. As used herein, the terms “homologous” and “homology” are interchangeable with the terms “identity” and “identical”.

The phrases “corresponding to” and “corresponds to” refer to the comparison between the nucleotide sequence of the oligonucleotide (i.e., the nucleobase or base sequence) or contiguous nucleotide sequence and the equivalent contiguous nucleotide sequence of a further sequence selected from either, (i) a sub-sequence of the reverse complement of the nucleic acid target, such as the mRNA which encodes the ATXN3 protein, and/or (ii) the sequence of nucleotides provided herein such as the group consisting of SEQ ID NOS: 15, 16, 17, 18, 19 or 20, or sub-sequence thereof. Nucleotide analogues are compared directly to their equivalent or corresponding nucleotides. A first sequence which corresponds to a further sequence under (i) or (ii) typically is identical to that sequence over the length of the first sequence (such as the contiguous nucleotide sequence) or, as described herein may, in some embodiments, be at least 80% homologous to a corresponding sequence, such as at least 85%, at least 90%, at least 91%, at least 92% at least 93%, at least 94%, at least 95%, at least 96% homologous, such as 100% homologous (identical).

Once one or more target sites have been identified, oligonucleotides are chosen which are sufficiently complementary to the target (i.e., hybridize sufficiently well and with sufficient specificity, to give the desired effect). For example, upon identifying a region of ATXN3 mRNA to target, oligonucleotides may be chosen based upon complementarity to the mRNA target or alternatively to the DNA encoding such mRNA target. In this context, “hybridization” means hydrogen bonding, which may be Watson-Crick, Hoogsteen or reversed Hoogsteen hydrogen bonding, between complementary nucleoside or nucleotide bases. For example, adenine (A) and thymine (T) are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. The sequence of an antisense compound may be, for example, about 40%, 50%, 60%, 65%, 70%, 75%, 80%, 85%, 90%, 95%, 97%, 97.5%, 99% or 100% complementary to that of its target sequence to be specifically hybridizable. An antisense compound is specifically hybridizable when binding of the compound to the target DNA or RNA molecule interferes with the normal function of the target DNA or RNA to cause a loss of function or utility, and there is a sufficient degree of complementarity to avoid non-specific binding of the antisense compound to non-target sequences under conditions in which specific binding is desired, (e.g., under physiological conditions in the case of in vivo assays or therapeutic treatment, and in the case of in vitro assays, under conditions in which the assays are performed). The phrases “reverse complement”, “reverse complementary” and “reverse complementarily” as used herein refer to an oligonucleotide that can hybridize with another given nucleic acid sequence on the same strand of a given nucleic acid molecule because of it's complementary relative to such nucleic acid sequence. For example, if the base in the 5′ to 3′ target nucleic acid strand is C, then the corresponding base in the 3′ to 5′ strand is G.

Antisense and other oligonucleotides of the invention which hybridize to the target nucleic acids (e.g., mRNA encoding a mutated ATXN3 protein) and inhibit expression of the target nucleic acid are identified through experimentation, and the sequences of these compounds are herein identified as preferred embodiments of the invention (e.g., the sequences identified in Table 1). The target nucleic acids or sites to which these preferred sequences are complementary are herein referred to as “active sites” and are therefore preferred sites for targeting (e.g., target sequences identified in Table 1). An example of an active site contemplated by the present invention includes the regions that surround the G987C SNP. Therefore another embodiment of the invention encompasses compounds which hybridize to this active site region, which can include nucleotides immediately upstream and/or downstream from the active site. For example, the region measuring about 1, 2, 5, 10, 12, 20, 30, 50, 60, 75, 80, 100 or more codons upstream and/or downstream from the G987C mutation.

The phrases “corresponding nucleotide analogue” and “corresponding nucleotide” are intended to indicate that the nucleobase in the nucleotide analogue and the naturally occurring nucleotide are identical. As such, in certain embodiments the nucleotide analogue will pair or hybridize with the corresponding nucleotides based on Watson-Crick base pairing principles. For example, when the 2-deoxyribose unit of the nucleotide is linked to an adenine, the corresponding nucleotide analogue contains a pentose unit (different from 2-deoxyribose) linked to an adenine and such nucleotide analogue would pair or hybridize with the corresponding thymine base.

### Length

The oligonucleotides may comprise or consist of a contiguous nucleotide sequence of a total of 5, 6, 7, 8, 9, 10, 11, 12, 13, 14, 15, 16, 17, 18, 19, 20, 21, 22, 23, 24, 25, 26, 27, 28, 29 or 30 contiguous nucleotides in length. In some embodiments, the oligonucleotides comprise or consist of a contiguous nucleotide sequence of a total of from about 8-25, such as about 10-22, such as about 12-18, such as about 13-17 or 12-16, such as about 13, 14, 15, 16 contiguous nucleotides in length. In some embodiments, the oligonucleotides comprise or consist of a contiguous nucleotide sequence of a total of 8, 9, 10, 11, 12, 13, or 14 contiguous nucleotides in length. In some embodiments, the oligonucleotide according to the invention consists of no more than 22 nucleotides, such as no more than 20 nucleotides, such as no more than 18 nucleotides, such as 15, 16 or 17 nucleotides. In some embodiments the oligonucleotide of the invention comprises less than 20 nucleotides. It should be understood that when a range is given for an oligonucleotide, or contiguous nucleotide sequence length it includes the lower an upper lengths provided in the range, for example from (or between) 10-30, includes both 10 and 30.

### Nucleosides and Nucleoside Analogues

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, as well as non-naturally occurring, synthetic or artificial nucleotides comprising modified or substituted sugar and/or base moieties, which are also referred to herein as “nucleotide analogues”. In certain embodiments, a nucleotide analogue may include oligonucleotides wherein both the sugar and the internucleoside linkage of the nucleotide units are replaced with novel groups, such as, for example, one or more peptide nucleic acids (PNA). PNA are generally characterized as having the sugar-backbone of an oligonucleotide replaced with an amide containing backbone, in particular an aminoethylglycine backbone. The nucleobases are retained and are bound directly or indirectly to aza nitrogen atoms of the amide portion of the backbone.

Herein, a single nucleotide unit may also be referred to as a monomer or nucleic acid unit.

In the field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligonucleotide. In the field of biotechnology, the term “nucleotide” is generally used to refer to a nucleic acid monomer or unit, and as such in the context of an oligonucleotide may refer to the base, such as the phrase “nucleotide sequence” typically refers to the nucleobase sequence (i.e. the presence of the sugar backbone and internucleoside linkages are implicit). Likewise, particularly in the case of oligonucleotides where one or more of the internucleoside linkage groups are modified, the term “nucleotide” may refer to a “nucleoside”, for example, the term “nucleotide” may be used, even when specifying the presence or nature of the linkages between the nucleosides.

As one of ordinary skill in the art would recognize, the 5′ terminal nucleotide of an oligonucleotide does not comprise a 5′ internucleotide linkage group, although it may or may not comprise a 5′ terminal group.

Non-naturally occurring nucleotides include nucleotides which have modified sugar moieties, such as bicyclic nucleotides or 2′ substituted nucleotides.

In some embodiments, the terms “nucleoside analogue” and “nucleotide analogue” are used interchangeably. “Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, (e.g., have no functional effect on the way the oligonucleotide works to inhibit target gene expression). Such equivalent analogues may nevertheless be useful if, for example, they are easier or cheaper to manufacture, or are more stable to storage or manufacturing conditions, or represent a tag or label. Preferably, however, the analogues will have a functional effect on the way in which the oligonucleotide functions to inhibit expression (e.g., by producing increased binding affinity to the target and/or increased resistance to intracellular nucleases and/or increased ease of transport into the cell). Specific examples of nucleoside analogues are described by, for example, in Freier, et al., Nucl. Acid Res. (1997) 25: 4429-4443 and Uhlmann, et Curr. Opinion in Drug Development (2000) 3(2): 293-213, and below:

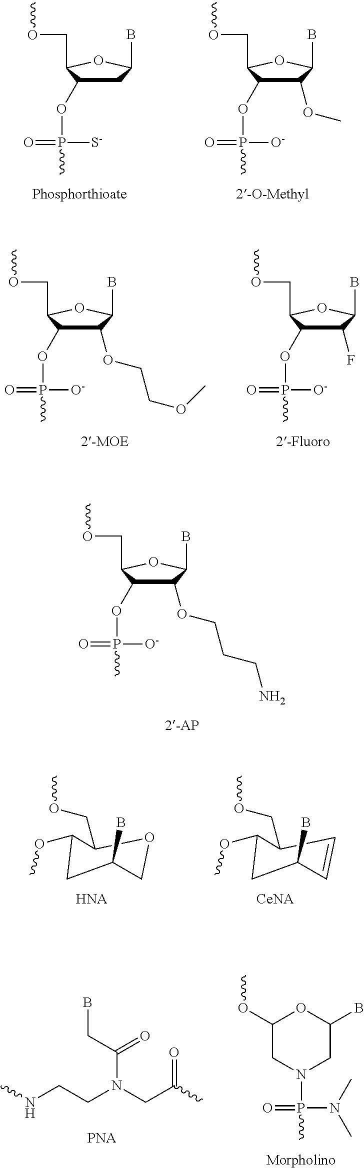

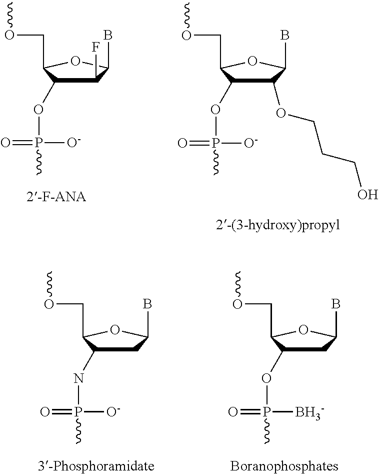

The oligonucleotides disclosed herein may thus comprise or consist of a simple sequence of naturally-occurring nucleotides, for example, preferably 2′-deoxynucleotides (referred to here generally as “DNA”), but also ribonucleotides (referred to here generally as “RNA”), or a combination of such naturally occurring nucleotides and one or more non-naturally occurring nucleotides, (e.g., nucleotide analogues). Such nucleotide analogues may suitably enhance the affinity of the oligonucleotide for the target sequence. Examples of suitable and preferred nucleotide analogues are provided in International Patent Application WO 2007/031091, or are referenced therein.

The incorporation of affinity-enhancing nucleotide analogues, such as locked nucleic acids (LNA) or 2′-substituted sugars, into the oligonucleotides can allow the size of the specifically binding oligonucleotide to be reduced, and may also reduce the upper limit to the size of the oligonucleotide before non-specific or aberrant binding takes place. Accordingly, in some embodiments, the oligonucleotide comprises at least 1 nucleoside analogue. In some embodiments the oligonucleotide comprises at least 2 nucleotide analogues. In some embodiments, the oligonucleotide comprises from 3-8 nucleotide analogues, (e.g., 6 or 7 nucleotide analogues). In certain embodiments, at least one of said nucleotide analogues is a LNA, for example at least 3 or at least 4, or at least 5, or at least 6, or at least 7, or at least 8 of the nucleotide analogues may be LNA. In some embodiments all the nucleotides analogues of the oligonucleotide may be LNA.

It will be recognized that when referring to a preferred nucleotide sequence motif or nucleotide sequence, which consists of only nucleotides, the oligonucleotides of the invention which are defined by that sequence may comprise or consist of a corresponding nucleotide analogue in place of one or more of the nucleotides present in such sequence, such as LNA units or other nucleotide analogues, which raise the melting temperature (Tₘ) of dissociation or duplex stability/Tₘ of the oligonucleotide/target duplex (i.e. affinity enhancing nucleotide analogues). As used herein, the term “Tₘ” refers to melting temperature and is used with reference to the temperature at which a population of complementary duplexed nucleic acid molecules (e.g., an antisense oligonucleotide and the corresponding mRNA target sequence) becomes half dissociated into single strands. A higher Tₘ is generally indicative of a more stable duplex.

In some embodiments, any mismatches between the nucleotide sequence of the oligonucleotide and the target sequence are preferably found in regions outside the affinity enhancing nucleotide analogues, such as region B as referred to herein, and/or region D as referred to herein, and/or at the site of non-modified nucleotides, such as DNA nucleotides, in the oligonucleotide, and/or in regions which are 5′ or 3′ to the contiguous nucleotide sequence.

Examples of such modifications of the nucleotides include modifying the sugar moiety to provide a 2′-substituent group or to produce a bridged (LNA) structure which enhances binding affinity and may also provide increased nuclease resistance. In some embodiments, a preferred nucleotide analogue is a LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligonucleotide of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid units as discussed by Christensen, et al., Nucl. Acids. Res. (2002) 30: 4918-4925) and 2′MOE units. In some embodiments there is only one of the above types of nucleotide analogues present in the oligonucleotide of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligonucleotide according to the invention comprises at least one locked nucleic acid (LNA) unit, such as 1, 2, 3, 4, 5, 6, 7, or 8 LNA units, such as from about 3-7 or 4-8 LNA units, or 3, 4, 5, 6 or 7 LNA units. In some embodiments, all the nucleotide analogues are LNA. In some embodiments, the oligonucleotide may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′ methylcytosine. In some embodiments of the invention, the oligonucleotide may comprise both LNA and DNA units. Preferably the combined total of LNA and DNA units is about 8-25, such as 10-24, preferably 10-20, such as 10-18, even more preferably 12-16. In some embodiments of the invention, the nucleotide sequence of the oligonucleotide, such as the contiguous nucleotide sequence consists of or comprises at least one LNA and the remaining nucleotide units are DNA units. In some embodiments the oligonucleotide comprises only LNA nucleotide analogues and naturally occurring nucleotides (such as RNA or DNA, most preferably DNA nucleotides), optionally with modified internucleotide linkages such as phosphorothioate.

As used herein, the term “nucleobase” refers to the base moiety of a nucleotide and covers both naturally occurring a well as non-naturally occurring variants. Thus, the term “nucleobase” covers not only the known purine and pyrimidine heterocycles but also heterocyclic analogues and tautomeres thereof. Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine. In some embodiments, at least one of the nucleobases present in the oligonucleotide is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In certain embodiments, the present invention relates to an antisense oligonucleotide comprising one or more C5-methylcytosine nucleobases. For example, an oligonucleotide comprising SEQ ID NO: 12, wherein the oligonucleotide comprises at least one nucleotide analogue at one or more nucleotides selected from the group consisting of: (i) the adenine nucleotide at one or more of positions 1 and 3 is an oxy-LNA; (ii) the guanine nucleotide at position 10 is an oxy-LNA; (iii) the cytosine nucleotide at one or more of positions 9 and 11 is an oxy-LNA; and (iv) the thymine nucleotide at position 2 is an oxy-LNA.

### Locked Nucleic Acids

As used herein, the term “LNA” refers to a bicyclic nucleoside analogue, known as a locked nucleic acid. It may refer to an LNA monomer, or, when used in the context of an “LNA oligonucleotide”, LNA may refer to an oligonucleotide containing one or more such bicyclic nucleotide analogues. LNA are characterised by the presence of a linker group (such as a bridge) between C2′ and C4′ of the ribose sugar ring, for example, as shown as the biradical R⁴*—R²* as described below.

The LNA used in the oligonucleotide compounds of the invention preferably have the structure of the general Formula I.

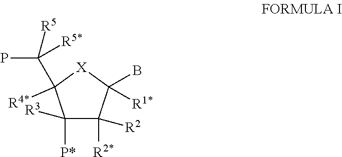

wherein for all chiral centers, asymmetric groups may be found in either R or S orientation;

wherein X is selected from —O—, —S—, —N(R^{N}*)—, —C(R⁶R⁶*)—, such as, in some embodiments —O—;

wherein B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands or is preferably a nucleobase or nucleobase analogue;

wherein P designates an internucleotide linkage to an adjacent monomer, or a 5′-terminal group, such internucleotide linkage or 5′-terminal group optionally including the substituent R⁵ or equally applicable the substituent R⁵*;

wherein P* designates an internucleotide linkage to an adjacent monomer, or a 3′-terminal group;

wherein R⁴* and R²* together designate a bivalent linker group consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-aminocarbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

wherein each of the substituents R¹*, R², R³, R⁵, R⁵*, R⁶ and R⁶*, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkylamino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof. For all chiral centers, asymmetric groups may be found in either R or S orientation.

In some embodiments, R⁴* and R²* together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and C₁₋₆alkyl, such as methyl, such as hydrogen.

In some embodiments, R⁴* and R²* together designate the biradical —O—CH(CH)OCH₃)— (2′O-methoxyethyl bicyclic nucleic acid—Seth at al., 2010, J. Org. Chem)—in either the R- or S-configuration.

In some embodiments, R⁴* and R²* together designate the biradical —O—CH(CH₂CH₃)— 2′O-ethyl bicyclic nucleic acid in either the R- or S-configuration.

In some embodiments, R⁴* and R²* together designate the biradical —O—CH(CH₃)— in either the R- or S-configuration. In some embodiments, R⁴* and R²* together designate the biradical —O—CH₂—O—CH₂—.

In some embodiments, R⁴* and R²* together designate the biradical —O—NR—CH₃—.

In some embodiments, the LNA units have a structure selected from the following group:

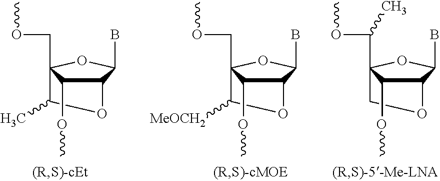

In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R¹*, R², R³, R⁵, R⁵* are hydrogen.

In some embodiments, R¹*, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R¹*, R², R³ are hydrogen.

In some embodiments, R⁵ and R⁵* are each independently selected from the group consisting of H, —CH₃, —CH₂—CH₃, —CH₂—O—CH₃, and —CH═CH₂. Suitably in some embodiments, either R⁵ or R⁵* are hydrogen, where as the other group (R⁵ or R⁵* respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, OJ₁, SJ₁, NJ₁J₂, N₃, COOJ₁, CN, O—C(═O)NJ₁J₂, N(H)C(═NH)NJ, J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ aminoalkyl, substituted C₁₋₆ aminoalkyl or a protecting group. In some embodiments either R⁵ or R⁵* is substituted C₁₋₆ alkyl. In some embodiments either R⁵ or R⁵* is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R⁵* is methyl, ethyl or methoxymethyl. In some embodiments either R⁵ or R⁵* is methyl. In a further embodiment either R⁵ or R⁵* is ethylenyl. In some embodiments either R⁵ or R⁵* is substituted acyl. In some embodiments either R⁵ or R⁵* is C(—O)NJ₁J₂. For all chiral centers, asymmetric groups may be found in either R or S orientation. Such 5′ modified bicyclic nucleotides are disclosed in International Patent Application WO 2007/134181.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyluracil, 2′thio-thymine, 5-methyl cytosine, 5-thiozolo-cytosine, 5-propynyl-cytosine, and 2,6-diaminopurine.

In some embodiments, R⁴* and R²* together designate a biradical selected from —C(R^{a}R^{b})—O—, —C(R^{a}R^{b})—C(R^{c}R^{d})—O—, C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})— C(R^{c}R^{d})—, —C(R^{a}R^{b})— C(R^{c}R^{d})— C(R^{e}R^{f})—, —C(R^{a})—C(R^{b})—C(R^{c}R^{d})—, —C(R^{a}R^{b})—N(R^{c})—, —C(R^{a}R^{b})—C(R^{c}R^{d})— N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{d}, R^{e}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R⁴* and R²* together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH—CH— For all chiral centers, asymmetric groups may be found in either R or S orientation.

In some embodiments, R⁴* and R²* together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, aminoalkyl or substituted C₁₋₆ aminoalkyl, such as hydrogen, and; wherein R^{c} is selected from the group consisting of hydrogen, halogen, C₁₋₆ alkyl, substituted C₁₋₆alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* together designate the biradical C(R^{a}R^{b})—O-consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆ alkoxy. In some embodiments Z is CH₃OCH₂—. For all chiral centers, asymmetric groups may be found in either R or S orientation. Such bicyclic nucleotides are disclosed in U.S. Pat. No. 7,399,845. In some embodiments, R¹*, R², R³, R⁵, R⁵* are hydrogen. In some embodiments, R¹*, R², R³* are hydrogen, and one or both of R⁵, R⁵* may be other than hydrogen as referred to above and in International Patent Application WO 2007/134181.

In some embodiments, R⁴* and R²* together designate a biradical which comprise a substituted amino group in the bridge such as consist or comprise of the biradical —CH₂—N(R^{c})—, wherein R^{c} is C₁₋₁₂ alkyloxy. In some embodiments R⁴* and R²* together designate a biradical —Cq₃q₄-NOR —, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)NJ₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in International Patent Application WO 2007/134181. In some embodiments R⁴* and R²* together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, CN, C(═O)J₁, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂ and; each J₁ and J₂ is, independently, H, C1-C₆ alkyl, substituted C1-C₆ alkyl, C₂-C₆ alkenyl, substituted C₂-C₆ alkenyl, C₂-C₆ alkynyl, substituted C₂-C₆ alkynyl, C1-C₆ aminoalkyl, substituted C1-C₆ aminoalkyl or a protecting group. Such compounds are disclosed in International Patent Application WO 2009/006478A.

In some embodiments, R⁴* and R²* form the biradical -Q-, wherein Q is C(q₁)(q₂)C(q₃)(q₄), C(q₁)═C(q₃), C[═C(q₁)(q₂)]-C(q₃)(q₄) or C(q₁)(q₂)—C[═C(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SO₂J₁, NJ₁J₂, N₃, CN, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(—S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R¹*, R², R³, R⁵, R⁵* are hydrogen. For all chiral centers, asymmetric groups may be found in either R or S orientation. Such bicyclic nucleotides are disclosed in WO/2008/154401. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R″, R², R³ are hydrogen and one or both of R⁵, R⁵* may be other than hydrogen as referred to above and in International Patent Applications WO/2007/134181 and WO2009/067647 (alpha-L-bicyclic nucleic acids analogs).

In some embodiments the LNA used in the oligonucleotide compounds of the invention preferably has the structure of the general Formula II.

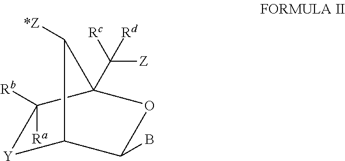

wherein Y is selected from the group consisting of —O—, —CH₂O—, —S—, —NH—, N(R^{e}) and/or —CH₂—;

wherein Z and Z* are independently selected among an internucleotide linkage, R^{H}, a terminal group or a protecting group;

wherein B constitutes a natural or non-natural nucleotide base moiety (nucleobase), and R^{H} is selected from hydrogen and C₁₋₄-alkyl; R^{a}, R^{b}R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino C₁₋₆-alkyl aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and wherein R^{H} is selected from hydrogen and C₁₋₄-alkyl. In some embodiments R^{a}, R^{b}R^{c}, R^{d} and R^{e} are, optionally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

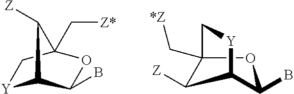

Specific exemplary LNA units are shown below:

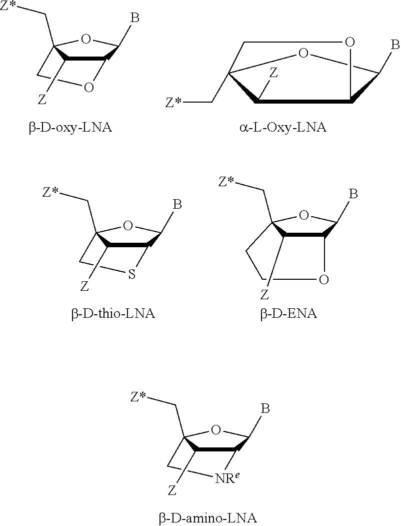

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration. In certain embodiments, the antisense oligonucleotides disclosed herein comprise at least one oxy-LNA. For example, disclosed herein is an oligonucleotide comprising SEQ ID NO: 12, wherein the oligonucleotide comprises at least one nucleotide analogue at one or more positions selected from the group consisting of: (a) the guanine nucleotide at position 1 is an oxy-LNA; (b) the adenine nucleotide at one or more of positions 2 and 3 is an oxy-LNA; (c) the cytosine nucleotide at one or more of positions 10 and 11 is an oxy-LNA; and (d) the thymine nucleotide at position 12 is an oxy-LNA. In some embodiments, some or all of such oxy-LNA are beta-D-oxy-LNA.

The term “ENA” comprises a locked nucleotide in which Y in the general formula above is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

### RNAse Recruitment

It is recognized that an oligonucleotide may function via non RNase-mediated degradation of target mRNA, such as by steric hindrance of translation, or other methods, however, the preferred oligonucleotides of the invention are capable of recruiting an endoribonuclease (RNase), such as RNase H.

It is preferable that the oligonucleotide, or contiguous nucleotide sequence, comprises of a region of at least 6, such as at least 7 consecutive nucleotide units, such as at least 8 or at least 9 consecutive nucleotide units, including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region B as referred to in the context of a gapmer as described herein. In some embodiments the size of the contiguous sequence which is capable of recruiting RNAse, such as region B, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. An oligonucleotide is deemed capable of recruiting RNaseH if, when provided with the complementary RNA target, it has an initial rate, as measured in pmol/l/min, of at least 1%, such as at least 5%, such as at least 10% or more than 20% of the of the initial rate determined using a DNA only oligonucleotide, having the same base sequence but containing only DNA monomers, with no 2′ substitutions, with phosphorothioate linkage groups between all monomers in the oligonucleotide, using the methodology provided by Example 91-95 of EP 1 222 309.

In some embodiments, an oligonucleotide is deemed essentially incapable of recruiting RNaseH if, when provided with the complementary RNA target, and RNaseH, the RNaseH initial rate, as measured in pmol/1/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligonucleotide is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligonucleotide which forms the consecutive nucleotide units which, when formed in a duplex with the complementary target RNA is capable of recruiting RNase consists of nucleotide units which form a DNA/RNA like duplex with the RNA target and include both DNA units and LNA units which are in the alpha-L configuration, particularly preferred being alpha-L-oxy LNA.

The oligonucleotides of the invention may comprise a nucleotide sequence which comprises both nucleotides and nucleotide analogues, and may be in the form of a gapmer, a headmer or a mixmer.

A “headmer” is defined as an oligonucleotide that comprises a region X and a region Y that is contiguous thereto, with the 5′-most monomer of region Y linked to the 3′-most monomer of region X. Region X comprises a contiguous stretch of non-RNase recruiting nucleoside analogues and region Y comprises a contiguous stretch (such as at least 7 contiguous monomers) of DNA monomers or nucleoside analogue monomers recognizable and cleavable by the RNase.

A “tailmer” is defined as an oligonucleotide that comprises a region X and a region Y that is contiguous thereto, with the 5′-most monomer of region Y linked to the 3′-most monomer of the region X. Region X comprises a contiguous stretch (such as at least 7 contiguous monomers) of DNA monomers or nucleoside analogue monomers recognizable and cleavable by the RNase, and region X comprises a contiguous stretch of non-RNase recruiting nucleoside analogues.

Other “chimeric” oligonucleotides, called “mixmers”, consist of an alternating composition of (i) DNA monomers or nucleoside analogue monomers recognizable and cleavable by RNase, and (ii) non-RNase recruiting nucleoside analogue monomers.

In some embodiments, in addition to enhancing affinity of the oligonucleotide for the target region, some nucleoside analogues also mediate RNase (e.g., RNaseH) binding and cleavage. Since α-L-LNA monomers recruit RNaseH activity to a certain extent, in some embodiments, gap regions (e.g., region B as referred to herein) of oligonucleotides containing α-L-LNA monomers consist of fewer monomers recognizable and cleavable by the RNaseH, and more flexibility in the mixmer construction is introduced.

### Gapmer Design

In some embodiments the oligonucleotide of the invention is a gapmer. A gapmer is an oligonucleotide which comprises a contiguous stretch of nucleotides which is capable of recruiting an RNAse, such as RNAseH, such as a region of at least 6 or 7 DNA nucleotides, referred to herein in as region B (B), wherein region B is flanked both 5′ and 3′ by regions of affinity enhancing nucleotide analogues, such as from about 1-6 nucleotide analogues 5′ and 3′ to the contiguous stretch of nucleotides which is capable of recruiting RNAse—these regions are referred to as regions A (A) and C (C) respectively.

In some embodiments, the monomers which are capable of recruiting RNAse are selected from the group consisting of DNA monomers, alpha-L-LNA monomers, C4′ alkylayted DNA monomers (see PCT/EP2009/050349 and Vester et al., Bioorg. Med. Chem. Lett. 18 (2008) 2296-2300), and UNA (unlinked nucleic acid) nucleotides (see Fluiter et al., Mol. Biosyst., (2009) 10: 1039). UNA is unlocked nucleic acid, typically where the C2—C3 C—C bond of the ribose has been removed, forming an unlocked “sugar” residue. Preferably the gapmer comprises a (poly)nucleotide sequence of formula (5′ to 3′), A-B-C, or optionally A-B-C-D or D-A-B-C, wherein; region A (A) (5′ region) consists or comprises of at least one nucleotide analogue, such as at least one LNA unit, such as from about 1-6 nucleotide analogues, such as LNA units, and; region B (B) consists or comprises of at least five consecutive nucleotides which are capable of recruiting RNAse (when formed in a duplex with a complementary RNA molecule, such as the mRNA target), such as DNA nucleotides, and; region C (C) (3′region) consists or comprises of at least one nucleotide analogue, such as at least one LNA unit, such as from 1-6 nucleotide analogues, such as LNA units, and; region D (D), when present consists or comprises of 1, 2 or 3 nucleotide units, such as DNA nucleotides.

In some embodiments, region A consists of 1, 2, 3, 4, 5 or 6 nucleotide analogues, such as LNA units, such as from about 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as from 2-5 nucleotide analogues, such as 2-5 LNA units, such as 3 or 4 nucleotide analogues, such as 3 or 4 LNA units.

In some embodiments B consists or comprises of 5, 6, 7, 8, 9, 10, 11 or 12 consecutive nucleotides which are capable of recruiting RNAse, or from 6-10, or from 7-9, such as 8 consecutive nucleotides which are capable of recruiting RNAse. In some embodiments region B consists or comprises at least one DNA nucleotide unit, such as 1-12 DNA units, preferably from 4-12 DNA units, more preferably from 6-10 DNA units, such as from about 7-10 DNA units, most preferably 8, 9 or 10 DNA units.

In some embodiments region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in International Application WO 2004/046160. International Application WO 2008/113832, which claims priority from U.S. provisional application 60/977,409, refers to ‘shortmer’ gapmer oligonucleotides. In some embodiments, oligonucleotides presented here may be such shortmer gapmers.

In some embodiments the oligonucleotide is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In some embodiments A consists of 1 LNA unit. In some embodiments A consists of 2 LNA units. In some embodiments A consists of 3 LNA units. In some embodiments C consists of 1 LNA unit. In some embodiments C consists of 2 LNA units. In some embodiments C consists of 3 LNA units. In some embodiments B consists of 7 nucleotide units. In some embodiments B consists of 8 nucleotide units. In some embodiments B consists of 9 nucleotide units. In certain embodiments, region B consists of 10 nucleoside monomers. In certain embodiments, region B comprises 1-10 DNA monomers. In some embodiments B comprises of from about 1-9 DNA units, such as 2, 3, 4, 5, 6, 7, 8 or 9 DNA units. In some embodiments B consists of DNA units. In some embodiments B comprises of at least one LNA unit which is in the alpha-L configuration, such as 2, 3, 4, 5, 6, 7, 8 or 9 LNA units in the alpha-L-configuration. In some embodiments B comprises of at least one alpha-L-oxy LNA unit or wherein all the LNA units in the alpha-L-configuration are alpha-L-oxy LNA units. In some embodiments the number of nucleotides present in A-B-C are selected from the group consisting of (nucleotide analogue units—region B—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In some embodiments the number of nucleotides in A-B-C are selected from the group consisting of: 2-7-1, 1-7-2, 2-7-2, 3-7-3, 2-7-3, 3-7-2, 3-7-4, and 4-7-3. In certain embodiments, each of regions A and C consists of three LNA monomers, and region B consists of 8 or 9 or 10 nucleoside monomers, preferably DNA monomers. In some embodiments both A and C consists of two LNA units each, and B consists of 8 or 9 nucleotide units, preferably DNA units. In various embodiments, other gapmer designs include those where regions A and/or C consists of 3, 4, 5 or 6 nucleoside analogues, such as monomers containing a 2%0-methoxyethyl-ribose sugar (2′-MOE) or monomers containing a 2′-fluoro-deoxyribose sugar, and region B consists of 8, 9, 10, 11 or 12 nucleosides, such as DNA monomers, where regions A-B-C have 3-9-3, 3-10-3, 5-10-5 or 4-12-4 monomers. Further gapmer designs are disclosed in International Application WO/2007/146511A2.

### Internucleotide Linkages

The monomers of the oligonucleotides described herein are coupled together via linkage groups. Suitably, each monomer is linked to the 3′ adjacent monomer via a linkage group. The person having ordinary skill in the art will understand that, in the context of the present invention, the 5′ monomer at the end of an oligonucleotide does not comprise a 5′ linkage group, although it may or may not comprise a 5′ terminal group.

The phrases “linkage group” and “internucleotide linkage” are intended to mean a group capable of covalently coupling together two nucleotides. Specific and preferred examples include phosphate groups and phosphorothioate groups. In certain embodiments, the antisense oligonucleotides disclosed herein have phosphorothioate internucleotide linkages at each internucleotide linkage (e.g., SEQ ID NOS: 19, 20, 21, 22 and 23). The nucleotides of the oligonucleotide of the invention or contiguous nucleotides sequence thereof are coupled together via linkage groups. Suitably each nucleotide is linked to the 3′ adjacent nucleotide via a linkage group.

Suitable internucleotide linkages include those listed within International Application WO 2007/031091, for example the internucleotide linkages listed on the first paragraph of page 34 of WO2007/031091.

It is, in some embodiments, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages may also be used for the flanking regions (A and C, and for linking A or C to D, and within region D, as appropriate).

Regions A, B and C, may however comprise internucleotide linkages other than phosphorothioate, such as phosphodiester linkages, particularly, for instance when the use of nucleotide analogues protects the internucleotide linkages within regions A and C from endo-nuclease degradation—such as when regions A and C comprise LNA nucleotides.

The internucleotide linkages in the oligonucleotide may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligonucleotide of the invention, the nucleotides and/or nucleotide analogues are linked to each other by means of phosphorothioate groups.

It is recognised that the inclusion of phosphodiester linkages, such as one or two linkages, into an otherwise phosphorothioate oligonucleotide, particularly between or adjacent to nucleotide analogue units (typically in region A and or C) can modify the bioavailability and/or bio-distribution of an oligonucleotide—see International Application WO 2008/053314

In some embodiments, such as the embodiments referred to above, where suitable and not specifically indicated, all remaining linkage groups are either phosphodiester or phosphorothioate, or a mixture thereof. In some embodiments all the internucleotide linkage groups are phosphorothioate.

When referring to specific gapmer oligonucleotide sequences, such as those provided herein it will be understood that, in various embodiments, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′methyl modified cytosine, in various embodiments, one or more of the Cs present in the oligonucleotide may be unmodified C residues.

### Oligonucleotides

The oligonucleotides of the invention may, for example, comprise a sequence selected from the group consisting of SEQ ID NOS: 9, 10, 11, 12, 13 and 14. In certain embodiments, the oligonucleotides of the invention may comprise a sequence selected from the group consisting of SEQ ID NO: 19, SEQ ID NO: 20, SEQ ID NO: 21, SEQ ID NO: 22 and SEQ ID NO: 23. In some embodiments, the oligonucleotides of the invention may, for example, be selected from the group consisting of the sequences identified in Tables 1 or 4.

### Conjugates

In the context of the present invention, the term “conjugate” is intended to indicate a heterogenous molecule formed by the covalent attachment of the oligonucleotide as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligonucleotide of the invention may comprise both a polynucleotide region which typically consists of a contiguous sequence of nucleotides, and a further non-nucleotide region. When referring to the oligonucleotide of the invention consisting of a contiguous nucleotide sequence, the compound may comprise non-nucleotide components, such as a conjugate component.

In various embodiments of the invention the oligonucleotide is linked to ligands/conjugates, which may be used, e.g. to increase the cellular uptake of oligonucleotides. International Application WO 2007/031091 provides suitable ligands and conjugates.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation may enhance the activity, cellular distribution or cellular uptake of the oligonucleotide of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligonucleotides of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example from about 1-50, such as 2-20 such as 3-10 amino acid residues in length, and/or polyalkylene oxide such as polyethylglycol (PEG) or polypropylene glycol (see e.g., International Application WO 2008/034123). Suitably the positively charged polymer, such as a polyalkylene oxide may be attached to the oligonucleotide of the invention via a linker such as the releasable inker described in WO 2008/034123.

By way of example, the following conjugate moieties may be used in the conjugates of the invention:

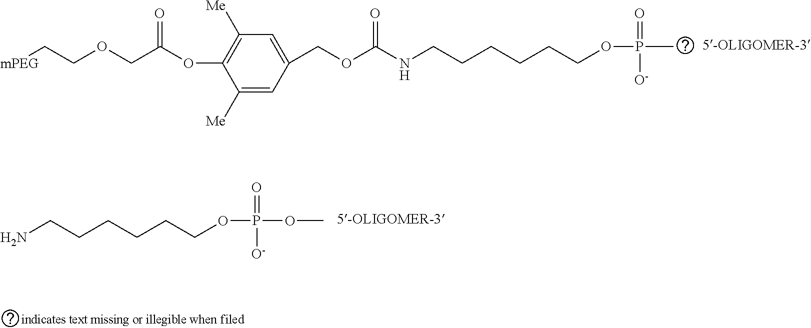

Activated oligonucleotides

The term “activated oligonucleotide,” as used herein, refers to an oligonucleotide of the invention that is covalently linked (i.e., functionalized) to at least one functional moiety that permits covalent linkage of the oligonucleotide to one or more conjugated moieties (i.e., moieties that are not themselves nucleic acids or monomers) to form the conjugates herein described. Typically, a functional moiety will comprise a chemical group that is capable of covalently bonding to the oligonucleotide via, for example, a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected (e.g., an NH₂ group). In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable (see e.g., U.S. Pat. No. 7,087,229).

In some embodiments, oligonucleotides of the invention are functionalized at the 5′ end in order to allow covalent attachment of the conjugated moiety to the 5′ end of the oligonucleotide. In other embodiments, oligonucleotides of the invention can be functionalized at the 3′ end. In still other embodiments, oligonucleotides of the invention can be functionalized along the backbone or on the heterocyclic base moiety. In yet other embodiments, oligonucleotides of the invention can be functionalized at more than one position independently selected from the 5′ end, the 3′ end, the backbone and the base.

In some embodiments, activated oligonucleotides of the invention are synthesized by incorporating during the synthesis one or more monomers that is covalently attached to a functional moiety. In other embodiments, activated oligonucleotides of the invention are synthesized with monomers that have not been functionalized, and the oligonucleotide is functionalized upon completion of synthesis. In some embodiments, the oligonucleotides are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligonucleotide via an ester group (—O—C(O)—(CH₂)_{w}NH).

In other embodiments, the oligonucleotides are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligonucleotide via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligonucleotides containing hindered esters as described above can be synthesized by any method known in the art, and in particular by methods disclosed in International Application WO 2008/034122 and the examples therein

In still other embodiments, the oligonucleotides of the invention are functionalized by introducing sulfhydryl, amino or hydroxyl groups into the oligonucleotide by means of a functionalizing reagent substantially as described in U.S. Pat. Nos. 4,962,029 and 4,914,210 (i.e., a substantially linear reagent having a phosphoramidite at one end linked through a hydrophilic spacer chain to the opposing end which comprises a protected or unprotected sulfhydryl, amino or hydroxyl group). Such reagents primarily react with hydroxyl groups of the oligonucleotide. In some embodiments, such activated oligonucleotides have a functionalizing reagent coupled to a 5′-hydroxyl group of the oligonucleotide. In other embodiments, the activated oligonucleotides have a functionalizing reagent coupled to a 3′-hydroxyl group. In still other embodiments, the activated oligonucleotides of the invention have a functionalizing reagent coupled to a hydroxyl group on the backbone of the oligonucleotide. In yet further embodiments, the oligonucleotide of the invention is functionalized with more than one of the functionalizing reagents as described in U.S. Pat. Nos. 4,962,029 and 4,914,210. Methods of synthesizing such functionalizing reagents and incorporating them into monomers or oligonucleotides are disclosed in U.S. Pat. Nos. 4,962,029 and 4,914,210.

In some embodiments, the 5′-terminus of a solid-phase bound oligonucleotide is functionalized with a dienyl phosphoramidite derivative, followed by conjugation of the deprotected oligonucleotide with, e.g., an amino acid or peptide via a Diels-Alder cycloaddition reaction.

In various embodiments, the incorporation of monomers containing 2′-sugar substitutions, such as a 2′-carbamate substituted sugar or a 2′-(O-pentyl-N-phthalimido)-deoxyribose sugar into the oligonucleotide facilitates covalent attachment of conjugated moieties to the sugars of the oligonucleotide. In other embodiments, an oligonucleotide with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. (See, e.g., Manoharan, et al., Tetrahedron Letters, (1991) 34:7171.)

In still further embodiments, the oligonucleotides of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligonucleotide synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.).

### Pharmaceutical Compositions

The oligonucleotides of the invention may be used in pharmaceutical formulations and compositions. Suitably, such compositions comprise a pharmaceutically acceptable solvent, such as water or saline, diluent, carrier, salt or adjuvant. PCT/DK2006/000512 provides suitable and preferred pharmaceutically acceptable diluent, carrier and adjuvants. Suitable dosages, formulations, administration routes, compositions, dosage forms, combinations with other therapeutic agents, pro-drug formulations are also provided in PCT/DK2006/000512.

The present invention also includes pharmaceutical compositions and formulations which include the oligonucleotides of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial (e.g., intrathecal, intracerebroventricular or intraventricular administration). Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999.

Compositions and formulations for oral administration include powders or granules, microparticulates, nanoparticulates, suspensions or solutions in water, saline or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAF-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. No. 08/886,829 (filed Jul. 1, 1997), U.S. Ser. No. 09/108,673 (filed Jul. 1, 1998), U.S. Ser. No. 09/256,515 (filed Feb. 23, 1999), U.S. Ser. No. 09/082,624 (filed May 21, 1998) and U.S. Ser. No. 09/315,298 (filed May 20, 1999).

Compositions and formulations for parenteral, intrathecal, intracerebroventricular or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances, which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

In another aspect, methods are provided to target a compound of the invention to a specific tissue, organ or location in the body. Exemplary targets include the cells and tissues of the central nervouse system (e.g., brain cells and tissues, neuronal cells, Purkinje cells, Schwann cells, oligodendrocytes and astrocytes). Methods of targeting compounds are well known in the art. In one embodiment, the compound is targeted by direct or local administration. For example, when targeting a blood vessel, the compound is administered directly to the relevant portion of the vessel from inside the lumen of the vessel, e.g., single balloon or double balloon catheter, or through the adventitia with material aiding slow release of the compound, e.g., a pluronic gel system as described by Simons et al., Nature (1992) 359: 67-70. Other slow release techniques for local delivery of the compound to a vessel include coating a stent with the compound. Methods of delivery of the oligonucleotides to a blood vessel are disclosed in U.S. Pat. No. 6,159,946.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate]derivatives according to the methods disclosed in International Applications WO 1993/24510 and WO 1994/26764 and in U.S. Pat. No. 5,770,713.

The term “pharmaceutically acceptable salts” refers to physiologically and pharmaceutically acceptable salts of the compounds of the invention (i.e., salts that retain the desired biological activity of the parent compound and do not impart undesired toxicological effects thereto). For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

### Applications

The oligonucleotides of the invention may be utilized as research reagents for, for example, diagnostics, therapeutics and prophylaxis. In research, such oligonucleotides may be used to specifically inhibit the synthesis of expanded or otherwise mutated ATXN3 (typically by degrading or inhibiting the mRNA and thereby prevent protein formation) in cells and experimental animals thereby facilitating functional analysis of the target or an appraisal of its usefulness as a target for therapeutic intervention.

In diagnostics the oligonucleotides may be used to detect and quantitate ATXN3 expression in cell and tissues by northern blotting, in-situ hybridisation or similar techniques.

For therapeutics, an animal or a human, suspected of having a disease or disorder (e.g., SCA3) which can be treated by modulating the expression of ATXN3 is treated by administering oligonucleotides in accordance with this invention. Further provided are methods of treating a mammal, such as treating a human, suspected of having or being prone to a disease or condition, associated with expression of ATXN3 by administering a therapeutically or prophylactically effective amount of one or more of the oligonucleotides or compositions of the invention. The oligonucleotide, a conjugate or a pharmaceutical composition according to the invention is typically administered in an effective amount.

The invention also provides for the use of the compound or conjugate of the invention as described for the manufacture of a medicament for the treatment of a disorder as referred to herein, or for a method of the treatment of as a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

Also contemplated by the present inventions is the use of the oligonucleotides described herein (e.g., an oligonucleotide that hybridizes to a region of SEQ ID NO: 4 comprising a G987C single nucleotide polymorphism) as a medicament. Similarly, provided herein are uses of the oligonucleotides described herein (e.g., oligonucleotides that hybridize to mRNA encoding or adjacent to the ATXN3 poly-glutamine expansion tract) in or for the treatment of diseases such as SCA3.

### Medical Indications

The oligonucleotides and other compositions according to the invention can be used for the treatment of conditions associated with over expression or expression of mutated version of the ATXN3 (e.g., spinocerebellar ataxia 3). In some embodiments, the oligonucleotides and compositions disclosed herein may be used as a medicament. In other embodiments, the oligonucleotides provided herein are used in or for the treatment of diseases. For example, an oligonucleotide that hybridizes to a region of SEQ ID NO: 4 comprising a G987C single nucleotide polymorphism can be used for the treatment of spinocerebellar ataxia 3. The invention further provides use of a compound of the invention in the manufacture of a medicament for the treatment of a disease, disorder or condition as referred to herein.

Generally stated, one aspect of the invention is directed to methods of treating a mammal suffering from or susceptible to conditions associated with mutated, aberrant, expanded or otherwise abnormal ATXN3 (e.g., relating to the expression of expanded ATXN3), comprising administering to the mammal a therapeutically effective amount of an oligonucleotide targeted to the gene product of a mutated or naturally occurring variant of ATXN3 (e.g., mRNA encoding a mutated or expanded ATXN3) that comprises one or more LNA units. The disease or disorder, as referred to herein, may, in some embodiments be associated with a mutation in the ATXN3 gene or a gene whose protein product is associated with or interacts with ATXN3. Therefore, in some embodiments, the target mRNA is a mutated form of ATXN3 mRNA.

One aspect of the invention is directed to the use of an oligonucleotide or a conjugate for the preparation of a medicament for the treatment of a disease, disorder or condition as referred to herein.

The methods of the invention are preferably employed for treatment or prophylaxis against diseases caused by abnormal levels of ATXN3. Alternatively stated, in some embodiments, the invention is furthermore directed to a method for treating abnormal levels of ATXN3, said method comprising administering a oligonucleotide of the invention, or a conjugate of the invention or a pharmaceutical composition of the invention to a patient in need thereof.

The invention also relates to an oligonucleotide, a composition or a conjugate as defined herein for use as a medicament.

The invention further relates to use of a compound, composition, or a conjugate as defined herein for the manufacture of a medicament for the treatment of abnormal levels of ATXN3 or expression of mutant forms of ATXN3 (such as allelic variants, such as those associated with one of the diseases referred to herein).

Moreover, the invention relates to a method of treating a subject suffering from a disease or condition such as those referred to herein.

A patient who is in need of treatment is a patient suffering from or likely to suffer from the disease or disorder.

In some embodiments, the term “treatment” as used herein refers to both treatment of an existing disease (e.g. a disease or disorder as herein referred to), or prevention of a disease, (i.e., prophylaxis). It will therefore be recognized that treatment as referred to herein may, in some embodiments, be prophylactic.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of ATXN3 is treated by administering oligonucleotides in accordance with this invention. The oligonucleotides of the invention can be utilized in pharmaceutical compositions by adding an effective amount of an oligonucleotide to a suitable pharmaceutically acceptable diluent or carrier.

The antisense compounds of the invention are useful for research and diagnostics, because these compounds hybridize to nucleic acids encoding ATXN3, enabling sandwich and other assays to easily be constructed to exploit this fact. Hybridization of the oligonucleotides of the invention with a nucleic acid encoding ATXN3 can be detected by means known in the art. Such means may include conjugation of an enzyme to the oligonucleotide, radiolabelling of the oligonucleotide or any other suitable detection means. Kits using such detection means for detecting the level of ATXN3 protein or mRNA in a sample may also be prepared.

While certain compounds, compositions and methods of the present invention have been described with specificity in accordance with certain embodiments, the following examples serve only to illustrate the compounds of the invention and are not intended to limit the same. Each of the publications, reference materials, GenBank accession numbers and the like referenced herein to describe the background of the invention and to provide additional detail regarding its practice is hereby incorporated by reference in its entirety.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

### EXAMPLES

The following examples describe several oligonucleotides targeting a mutant ATXN3 mRNA transcript that comprises the 0987C single nucleotide polymorphism (SNP) located one nucleotide from the pathogenic (CAG)ₙ repeat, as well as various superior properties of these oligonucleotides. In particular, Example 1 demonstrates the efficacy of 20 oligonucleotides to knock-down expression of mutant/expanded ATXN3 and the selectivity of those 20 oligonucleotides (e.g., inhibition of mutant/expanded ATXN3 expression as compared to inhibition of wild-type ATXN3 expression). Example 2 demonstrates the strength of each of twelve oligonucleotides to inhibit expression of mutant/expanded ATXN3, measured as an IC₅₀ value, and the difference in the strength to inhibit mutant/expanded ATXN3 expression as compared to wild-type ATXN3 expression. Example 3 describes the binding energy, measured as melting temperature (Tₘ), between each of the twelve oligonucleotides and either the expanded G987C ATXN3 target sequence (perfect complementarity) or the wild-type ATXN3 sequence (one complementarity mismatch at the G987C SNP site). Example 4 demonstrates the nuclease sensitivity, measured as plasma stability, of the twelve oligonucleotides. Example 5 describes an assessment of the in vivo tolerance for selected oligonucleotides in a standard 16-day mouse study.

### Example 1

### Efficacy and Selectivity Testing for 20 Oligonucleotides

A total of 20 antisense oligonucleotides, each having one or more locked nucleic acids (LNA), were designed to selectively target the human ATXN3 mutated (i.e., expanded) allele. In particular, the 20 LNA antisense oligonucleotides were designed to selectively target and hybridize to a region of the expanded ATXN3 allele that includes a “C” at the position one nucleotide from the pathogenic (CAG)ₙ expansion of the ATXN3 mRNA (i.e., the G987C SNP) as well as the nucleotides upstream and/or downstream of that position.

To characterize each of the 20 oligonucleotides, HEK-293 cells were stably transfected with a reporter construct (pFLAG-ATXN3Q81-FL-FFLuciferase) that comprised the coding region of the mutated ATXN3 transcript that was characterized as having 81 (CAG) repeats and included the G987C SNP fused to a firefly luciferase transcript. Luciferase activity correlated to the amount of mutated ATXN3 protein which was used as the read-out and was normalized to cell proliferation and viability as measured by the WST-1 assay. The WST-1 assay is a colorimetric assay for quantification of cell proliferation and cell viability, based on the cleavage of the tetrazolium salt WST-1 by mitochondrial dehydrogenases in viable cells. A qualitative PCR (qPCR) assay was designed to specifically recognize the vector expression products by targeting the FLAG sequence. In addition, a qPCR assay was designed to specifically recognize the endogenously expressed ATXN3 product by targeting the 5′ UTR of ATXN3, which was not included as part of the vector expression system.

In order to investigate the effect of the antisense oligonucleotides on the expression of the wild-type and expanded/mutated ATXN3 the qPCR assays were optimized to specifically recognize expression of either the mutated or the wild-type (endogenous) ATXN3. With respect to the mutated ATXN3 reporter construct, a qPCR assay targeting the luciferase gene which was part of the ATXN3-reporter construct (located downstream of the ATXN3 transcript) was designed and optimized. With respect to determining the expression of the wild-type allele (that was endogenously expressed in the HEK-293 cells), a qPCR assay targeting the 5′ UTR of the endogenous ATXN3 transcript was designed and optimized, and could distinguish the mutant ATXN3 allele because the targeted 5′ UTR was not present in the reporter construct.

The efficacy and selectivity of each of the oligonucleotides was assessed in the pFLAG-ATXN3Q81-FL-FFLuciferase-transfected HEK-293 cells using gymnotic delivery (i.e., unassisted uptake) as a means of introducing the oligonucleotides into the cells by exposure to media having final concentrations of 1 μM, 5 μM and 25 μM. Specifically, the oligonucleotides were delivered by gymnosis, after which the cells were washed thoroughly to eliminate any oligonucleotide still adhering to the surface of the cell or otherwise remaining in the culture vessel. The cells were harvested and assayed by qPCR 48 hours after transfection and the percentage of mRNA expression was determined using the relevant qPCR assay. In all experiments an oligonucleotide designated PCON2 (a 16-mer gapmer targeting the firefly luciferase transcript) was included as positive control of mutant ATXN3 (reporter construct), an oligonucleotide designated PCON1 (a 16-mer gapmer targeting both mutated and wild-type ATXN3) was included as positive control, and an oligonucleotide designated NCON (a scrambled oligonucleotide) was included as negative control. The mock-treated samples were evaluated in the absence of a test oligonucleotide, which was replaced with water. The read out was the percentage of mRNA levels of the mutated ATXN3 transcript (reporter construct) and the endogenous ATXN3 transcript relative to mock-treated samples.

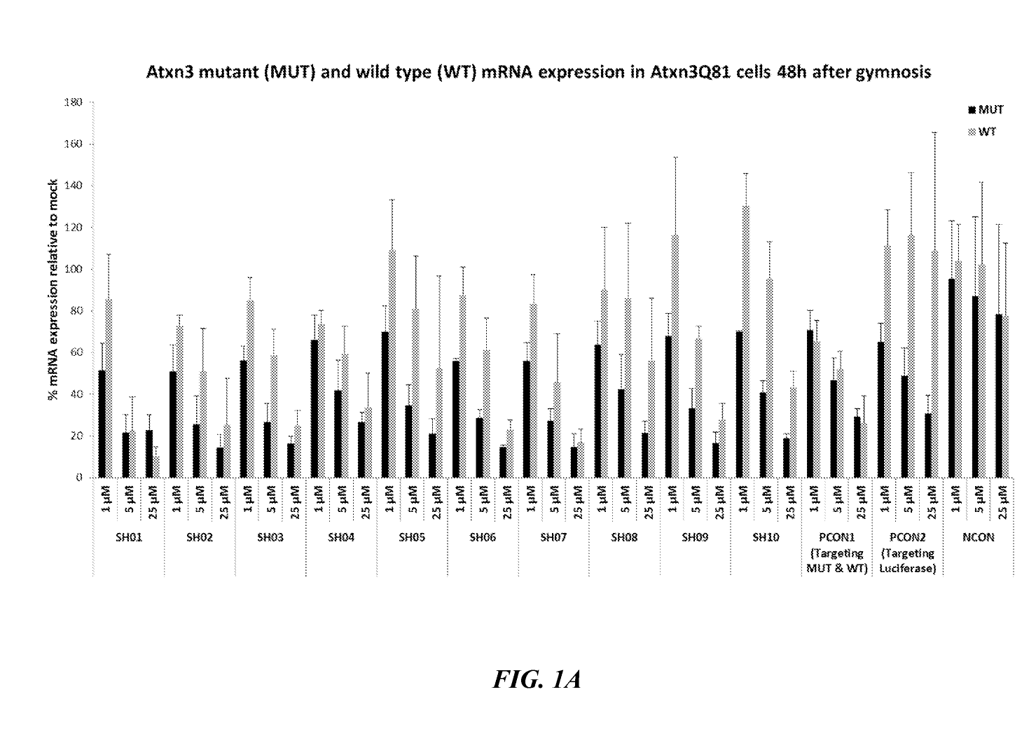

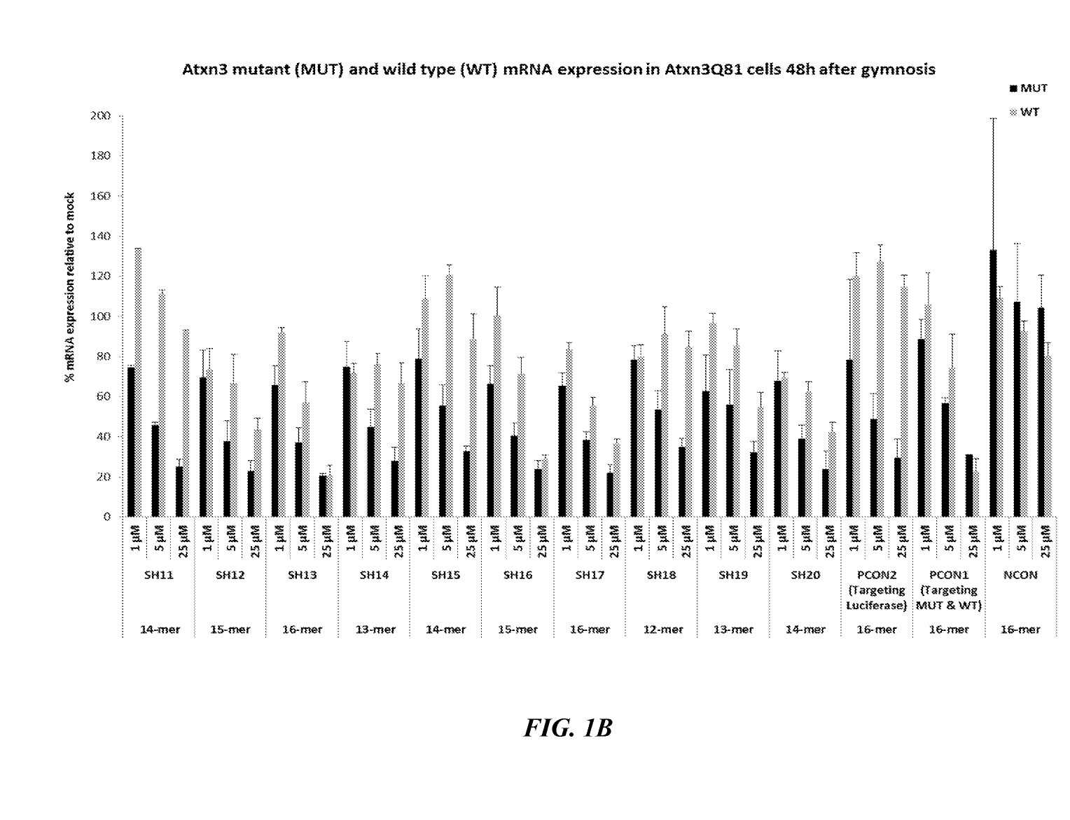

The results of the instant studies are reflected in FIGS. 1A and 1B as a percentage of mRNA expressed relative to the mock-treated samples. The 20 oligonucleotides are identified with identifiers, each starting with “SH”. A robust knock-down with marked dose response was observed for most of the oligonucleotides evaluated and, as illustrated in FIGS. 1A and 1B, and a clear ranking could be established. As illustrated in FIGS. 1A and 1B, certain oligonucleotides modulated (i.e., diminished or otherwise reduced) the expression of the wild-type ATXN3 and/or mutant ATXN3 by about 60% or less, 50% or less, 40% or less, 30% or less, 25% or less, 20% or less, and even 10% or less as compared to the expression of ATXN3 observed in the mock samples, depending on the concentration (1 μM, 5 μM or 25 μM) of the oligonucleotide. Moreover, certain oligonucleotides substantially and preferentially reduced expression (i.e., down-regulated expression) of the mutant ATXN3 as compared to the wild-type ATXN3 expression. For example, the oligonucleotide designated as SH10 (SEQ ID NO: 20) at a concentration of 1 μM diminished the mutant ATXN3 level to nearly half of the corresponding wild-type ATXN3 level. The positive control, designated as 5744, demonstrated substantial inhibition of both mutant and wild-type ATXN3 levels, showing little to no specificity because it is not directed to the mutation site.

The foregoing data illustrate that each of the 20 oligonucleotides showed efficacy and selectively at one or more of the concentrations evaluated. From that collective data twelve oligonucleotides were identified and selected for additional characterization.

### Example 2

### IC₅₀ Testing for Twelve Oligonucleotides

The twelve oligonucleotides selected as a result of the studies described in Example 1 were further evaluated for their inhibition strength by assessing the IC₅₀ value for each oligonucleotide. The selected oligonucleotide candidates were subjected to IC₅₀ determinations performed in the HEK-293 cells that had been stably transfected with the previously-described reporter construct (pFLAG-ATXN3Q81-FL-FFLuciferase).

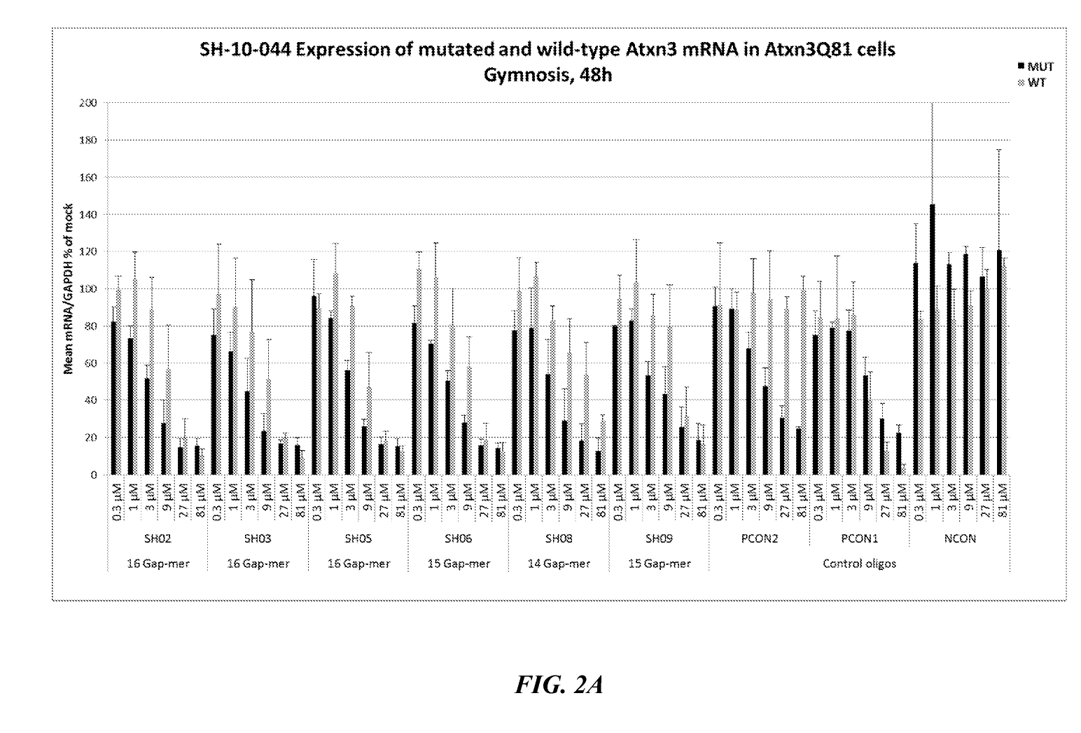

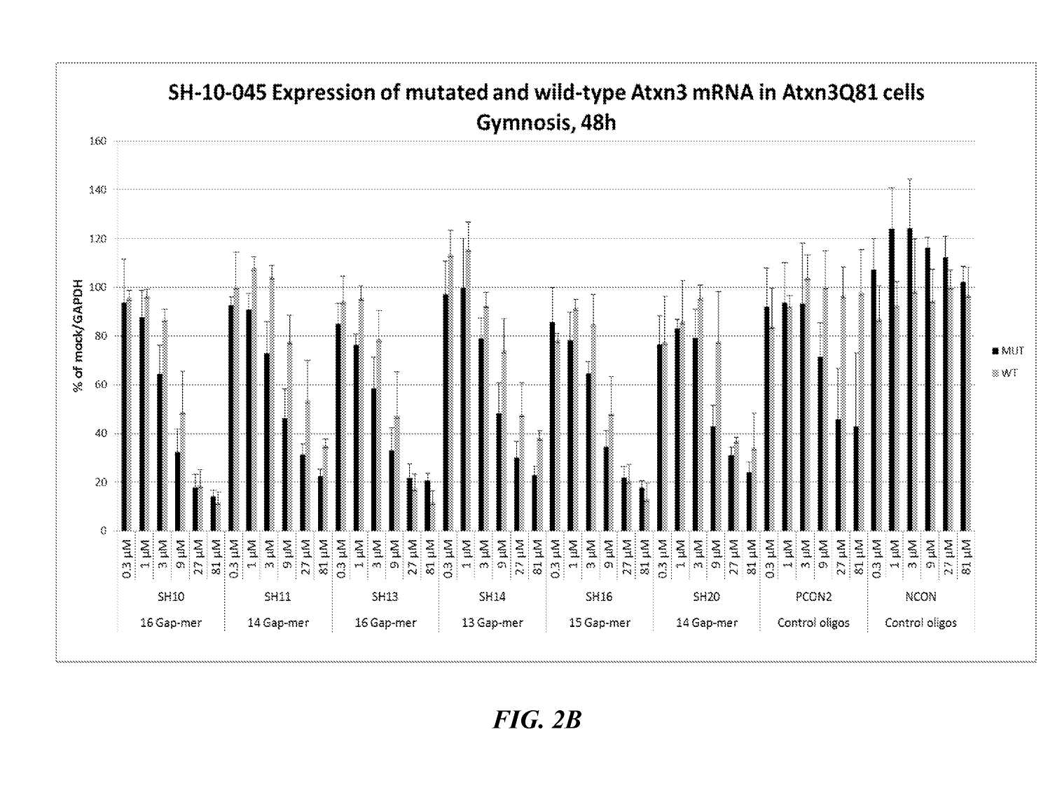

The oligonucleotides were introduced to the cells by gymnosis using media having final concentrations of 0.3 μM, 1 μM, 3 μM, 9 μM, 27 μM and 81 μM. The cells were subsequently harvested 48 hours after the addition of oligonucleotides and the mRNA of the mutated ATXN3 transcript (reporter construct) and the endogenous ATXN3 transcript were extracted and analyzed by qPCR. FIGS. 2A and 2B illustrate the expression of the mutated ATXN3 transcript (MUT) and the endogenous ATXN3 (WT) mRNA in the ATXN3-Q81 transfected cells following gymnotic delivery of the selected oligonucleotides. The results were normalized to the endogenous GAPDH levels and expressed as a percent of the mock treated samples. The studies were repeated three times and the values were plotted in Graphpad Prism against a sigmoidal response curve using non-linear regression.

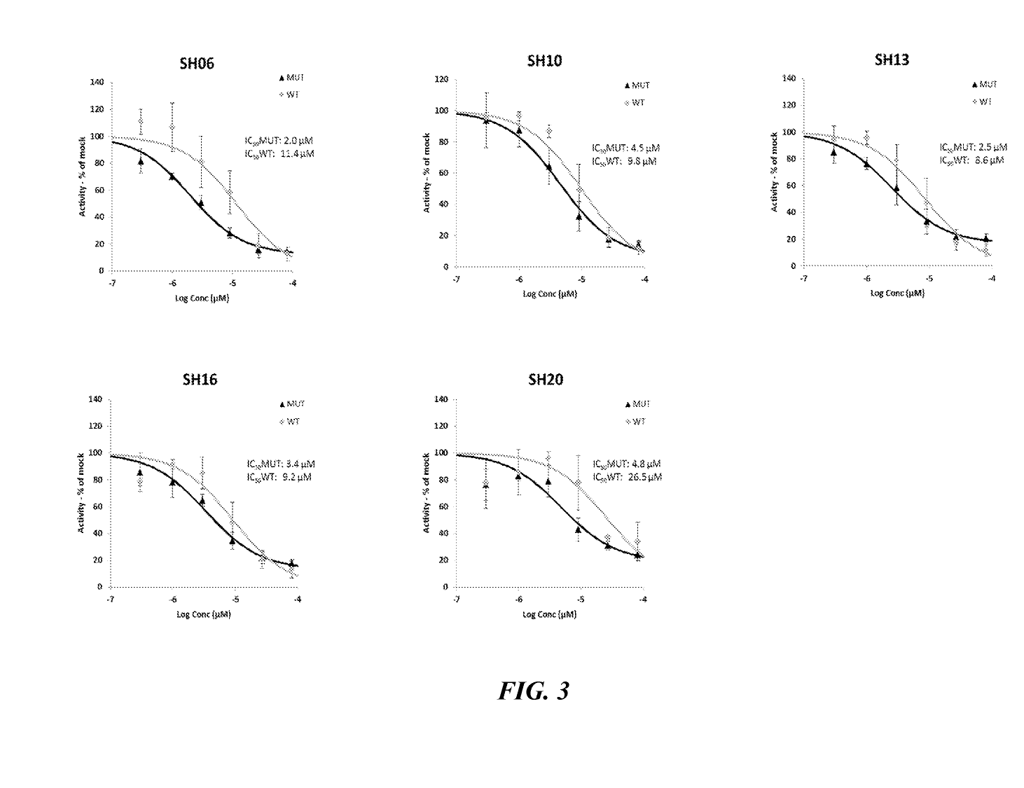

From the regression, an estimate of the IC₅₀ value in the reporter assay was determined. The IC₅₀ curves are shown in FIG. 3 and the corresponding IC₅₀ values are tabulated in Table 2 below. The selectivity of the twelve oligonucleotides calculated was calculated as the fold difference of the IC₅₀ value against the mutated ATXN3 (MUT) versus endogenous ATXN3 (WT) transcripts and the IC₅₀ values for the five oligonucleotides selected as leads are shown in Table 2 below.

TABLE 2

| | | | Selectivity (IC50 |
|---|---|---|---|
| | IC50 (MUT, | IC50 (WT, | WT/MUT) Gymnosis |
| SH No. | Gymnosis) μM | Gymnosis) μM | (fold) |
| SH06 | 2.0 | 11.4 | 5.7 |
| SH10 | 4.5 | 9.8 | 2.2 |
| SH13 | 2.5 | 8.6 | 3.4 |
| SH16 | 3.4 | 9.2 | 2.7 |
| SH20 | 4.8 | 26.5 | 5.5 |

With regard to the selectively of the selected oligonucleotides for the mutant/expanded ATXN3 reporter relative to the wild-type ATXN3 transcript, nine out of the twelve selected oligonucleotides demonstrated a better than approximately 3-fold selectivity for the mutant/expanded reporter over the wild-type transcript.

### Example 3

### Melting Temperature for Twelve Oligonucleotides Annealed to Wild-Type or Mutant ATXN3

As part of the characterization of the twelve oligonucleotide candidates described in Example 2 above, the melting temperature (Tₘ) of each oligonucleotide from a complementary mutant target RNA (designated cMUT) was evaluated. Each of the twelve oligonucleotides was also tested against an RNA representing the wild-type allele (designated cWT), which included a single complementarity mismatch against the G987C SNP located one nucleotide from the (CAG) expansion.

The Tₘ values for each of the twelve oligonucleotides were determined, and the change in Tₘ (ΔTₘ) represents the average of the melting and annealing temperature for each oligonucleotide. The Tₘ data representing the five selected lead oligonucleotides are presented in Table 3, where the ΔTm was defined as the difference between the cMUT (perfect complement) and cWT (one complementary mismatch at mutation site) values.

TABLE 3

| | RNA cMUT | RNA cWT | ΔTₘ |
|---|---|---|---|
| Oligo | Tₘ (° C.) | Tₘ (° C.) | (MUT − WT) (° C.) |
| SH06 | 76.8 | 65.9 | 10.9 |
| SH10 | 75.9 | 66.9 | 9.0 |
| SH13 | 77.8 | 68.0 | 9.8 |
| SH16 | 76.8 | 64.0 | 12.8 |
| SH20 | 69.2 | 60.2 | 9.0 |

When targeting the twelve oligonucleotides against the WT allele of ATXN3, the single mismatch is a G-C mismatch at the G987C SNP located one nucleotide 3′ to the pathogenic (CAG) expansion. The ΔTₘ ranged from a high of 15.5° C. to a low of negative 8.7° C., while for the five selected oligonucleotides, as illustrated in Table 3, the ΔTₘ ranged from a high of 12.8° C. to a low of 9.0° C.

### Example 4

### Plasma Stability for Each of Twelve Oligonucleotides

As part of the characterization of the twelve oligonucleotides described in Examples 2 and 3, the stability of each of the twelve oligonucleotides was assessed. Specifically, each of the twelve oligonucleotides was incubated in cerebrospinal fluid with added brain tissue for 120 hours at 37° C., and samples were taken and analyzed at 0, 24, 48, 96 and 120 hours. The samples were evaluated by SDS-PAGE for the loss of full length oligonucleotide and the emergence of degradation products. The samples were compared to an unstable, 18-mer full phosphorothioate positive control oligonucleotide designated PCON.

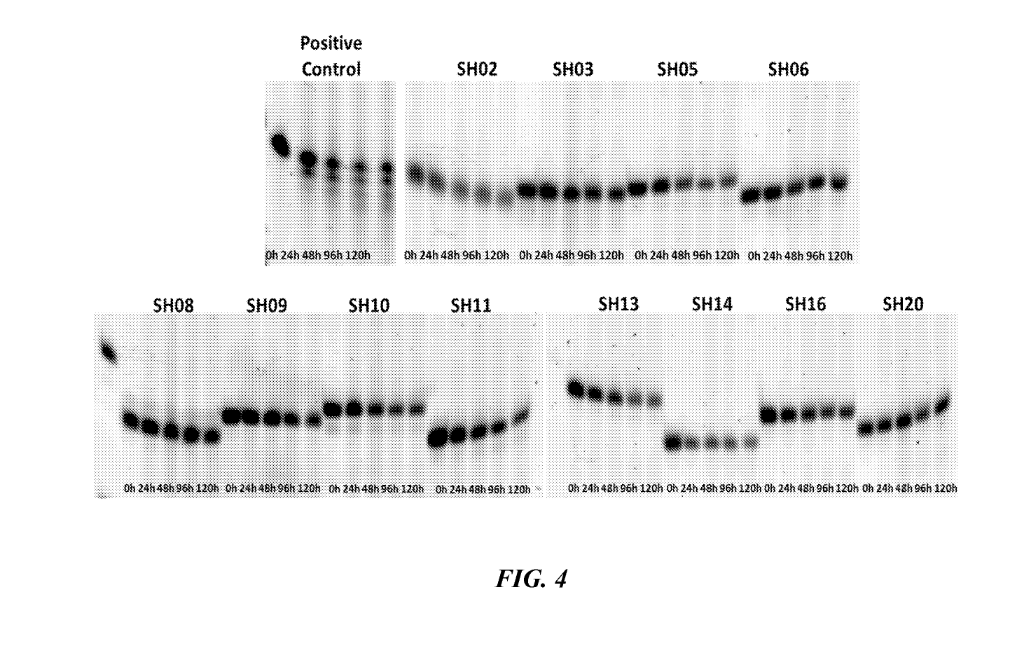

The plasma stability of all of the oligonucleotides was found to be within the expected ranges. As illustrated in FIG. 4, all of the oligonucleotides were found to have an overall half-life of greater than about 96 hours.

### Example 5

### In Vivo Tolerance Study for Oligonucleotides

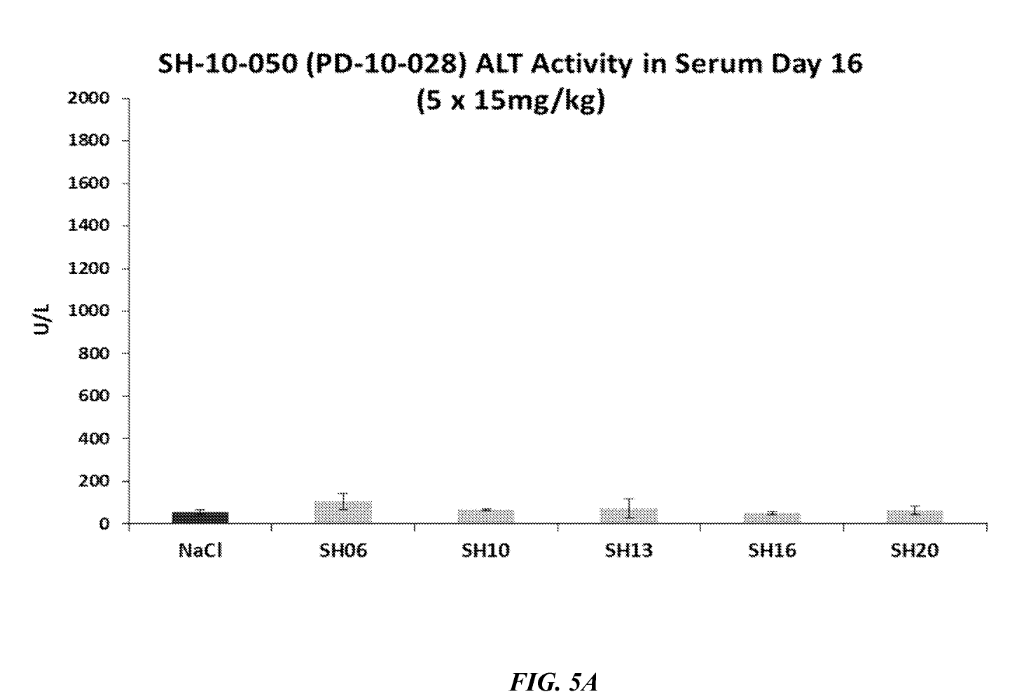

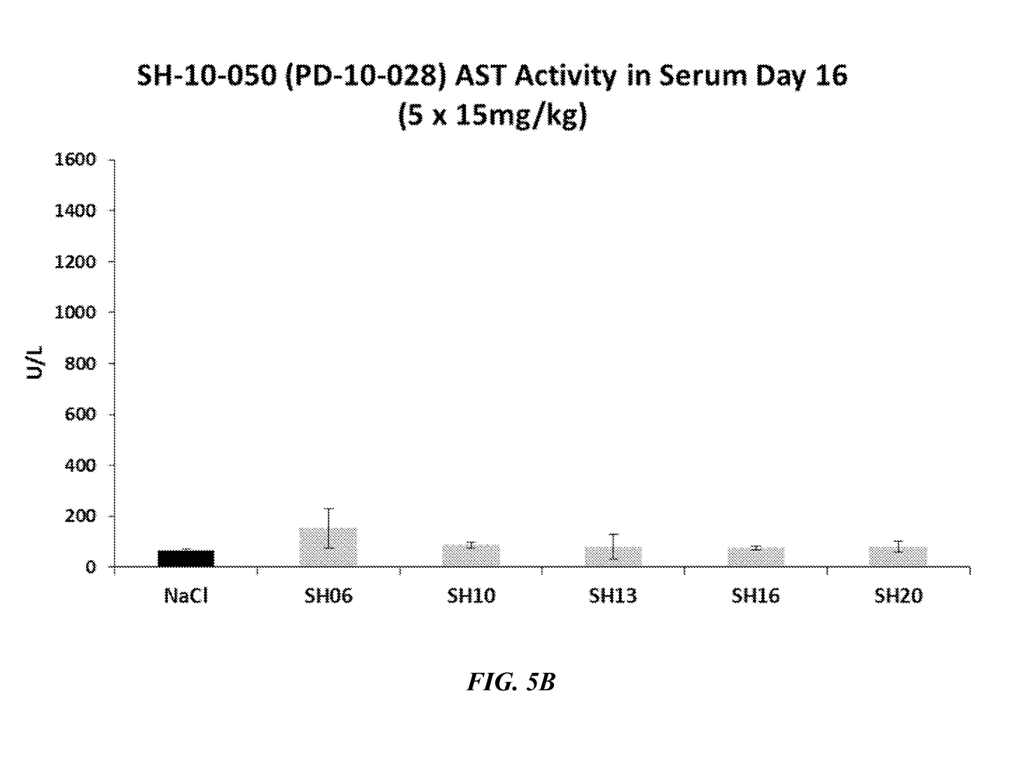

The in vivo tolerance of each of the twelve oligonucleotides was tested in a 16-day mouse study. Each of the oligonucleotides was tested for in vivo tolerance in female NMRI mice, primarily to assess any undesired liver effects. The subject animals were dosed at 15 mg/kg intravenously on days 0, 3, 7, 10 and 14, and then sacrificed on day 16. Mouse serum was sampled and analyzed for alanine aminotransferase (ALT) and aspartate aminotransferase (AST) concentrations. Control mice were administered a saline control. As depicted in FIGS. 5A and 5B, the oligonucleotides designated SH06 (SEQ ID NO: 19), SH10 (SEQ ID NO: 20), SH13 (SEQ ID NO: 21), SH16 (SEQ ID NO: 22) and SH20 (SEQ ID NO: 23) demonstrated a negligible elevation in the levels of liver enzymes relative to the saline control.

Based on the characteristics identified in Examples 1-4, the five oligonucleotides namely the oligonucleotides designated SH06, SH10, SH13, SH16 and SH20) were shown to have superior properties for use in administration to a subject to diminish the expression expanded (mutant) ATXN3 and were designated lead compounds. As described above, the selected oligonucleotides were selected as being superior relative to the population of unselected oligonucleotides. The sequences of these five lead oligonucleotides are set forth in Table 4 below, where β-D-oxy LNA are illustrated in bold capital letters with the superscript “o” the right, lowercase letters indicate deoxyriboses, and ‘s’ and ‘m’ correspond to phosphorothioate and C5-methylcytosine, respectively.

TABLE 4

| | | ATXN3 | | | |
|---|---|---|---|---|---|
| | | Target | | | |
| | | Nucleotide | | | |
| | SEQ ID | Position | | | |
| Identifier | NO: | NM_004993 | Length | OLIGONUCLEOTIDE SEQUENCE | mRNA Target Sequence |
| SH06 | 19 | 980-994 | 15 | 5′ - [symbol] gₛ gₛ tₛ cₛ cₛ | 5′ - AGCAGCGGGACCUAU - 3′ |
| | | | | ^{m}cₛ gₛ cₛ tₛ [symbol] - 3′ | (SEQ ID NO: 13) |
| SH10 | 20 | 981-996 | 16 | 5′ - [symbol] tₛ aₛ gₛ gₛ tₛ | 5′ - GCAGCGGGACCUAUCA - 3′ |
| | | | | cₛ cₛ ^{m}cₛ gₛ cₛ [symbol] - 3′ | (SEQ ID NO: 14) |
| SH13 | 21 | 982-997 | 16 | 5′ - [symbol] aₛ tₛ aₛ gₛ gₛ | 5′ - CAGCGGGACCUAUCAG - 3′ |
| | | | | tₛ cₛ cₛ ^{m}cₛ gₛ [symbol] - 3′ | (SEQ ID NO: 15) |
| SH16 | 22 | 983-997 | 15 | 5′ - [symbol] aₛ tₛ aₛ gₛ gₛ | 5′ - AGCGGGACCUAUCAG - 3′ |
| | | | | tₛ cₛ cₛ ^{m}cₛ [symbol] - 3′ | (SEQ ID NO: 16) |
| SH20 | 23 | 984-997 | 14 | 5′ - [symbol] gₛ aₛ tₛ aₛ gₛ gₛ | 5′ - GCGGGACCUAUCAG - 3′ |
| | | | | tₛ cₛ cₛ [symbol] - 3′ | (SEQ ID NO: 17) |

### Example 6

### Demonstration of Therapeutic Benefit of Oligonucleotides in Mouse Model of Spinocerebellar Ataxia 3

The ability of the lead oligonucleotides designated SH06, SH10, SH13, SH16 and SH20 (corresponding to SEQ ID NOS: 19, 20, 21, 22 and 23, respectively) to reduce disease pathology relating to the expression of expanded/mutated ATXN3 is determined as follows. An animal model of spinocerebellar ataxia 3 may be developed or selected. The preferred animal model correlates with the human mutation of ATXN3 (e.g., extended ATXN3 characterized as having the G987C SNP). Primary endpoints for the in-life efficacy can include, for example, improvement in gait and limb ataxia, dysarthria, pyramidal signs, dystonia, oculomotor disorders, faciolingual weakness, neuropathy, progressive sensory loss, lethargy and parkinsonian features. Disease phenotype, and reversal thereof by each oligonucleotide, can be verified by various methodologies known in the art, for example, by physical or histological examination.

Parameters to be investigated can include, among other things, an improvement in gait and limb ataxia, dysarthria, pyramidal signs, dystonia, oculomotor disorders, faciolingual weakness, neuropathy, progressive sensory loss, lethargy and parkinsonian features in oligonucleotide-treated mice that are statistically better than in oligonucleotide-untreated mice.

The studies described in this Example can provide further characterization of (1) antisense-locked nucleic acid oligonucleotides directed against mutant or expanded ATXN3 as an effective treatment of diseases SCA3 or Machado-Joseph disease, (2) oligonucleotides for use in particular spinocerebellar ataxia 3 disease models and as candidates for pharmacokinetics and toxicology studies and (3) dosing and dose-schedules for clinical administration of the oligonucleotides described herein.

### Sequences

| | | |
|---|---|---|
| SEQ ID NO: 1 | Wild-Type ATXN3 | NM_004993 |
| SEQ ID NO: 2 | Focused region of | 961 cagcagcagc agcagcagca |
| | WT ATXN3 | 981 gcagggggac ctatcaggac |
| | | 1001 agagttcaca tccatgtgaa |
| SEQ ID NO: 3 | More focused region | 977 agcagcaggg ggacctatca |
| | of WT ATXN3 | 997 g |
| SEQ ID NO: 4 | Mutant ATXN3 (G987C) | SNP ID rs12895357 |
| SEQ ID NO: 5 | Focused region of MUT | 961 cagcagcagc agcagcagca |
| | ATXN3 around nucleotides | 981 gcagcgggac ctatcaggac |
| | 961-1020 encoding G987C | 1001 agagttcaca tccatgtgaa |
| | mutation | |
| SEQ ID NO: 6 | More focused region of | 977 agcagcagcg ggacctatca |
| | MUT ATXN3 around | 997 g |
| | nucleotides 977-997 | |
| | encoding G987C mutation | |

| | | |
|---|---|---|
| SEQ ID NO: 7 | ATAGGTCCCGCTGCT | |
| SEQ ID NO: 8 | TGATAGGTCCCGCTGC | |
| SEQ ID NO: 9 | CTGATAGGTCCCGCTG | |
| SEQ ID NO: 10 | CTGATAGGTCCCGCT | |
| SEQ ID NO: 11 | CTGATAGGTCCCGC | |
| SEQ ID NO: 12 | ATAGGTCCCGC | |
| SEQ ID NO: 13 | AGCAGCGGGACCUAU | |
| SEQ ID NO: 14 | GCAGCGGGACCUAUCA | |
| SEQ ID NO: 15 | CAGCGGGACCUAUCAG | |
| SEQ ID NO: 16 | AGCGGGACCUAUCAG | |
| SEQ ID NO: 17 | GCGGGACCUAUCAG | |
| SEQ ID NO: 18 | GCGGGACCUAU | |
| SEQ ID NO: 19 | SH06 | 5′- [symbol] gₛ gₛ tₛ cₛ cₛ ^{m}cₛ gₛ cₛ tₛ [symbol] -3′ |
| SEQ ID NO: 20 | SH10 | 5′- [symbol] tₛ aₛ gₛ gₛ tₛ cₛ cₛ ^{m}cₛ gₛ cₛ [symbol] -3′ |
| SEQ ID NO: 21 | SH13 | 5 - [symbol] aₛ tₛ aₛ gₛ gₛ tₛ cₛ cₛ ^{m}cₛ gₛ [symbol] -3′ |
| SEQ ID NO: 22 | SH16 | 5′- [symbol] aₛ tₛ aₛ gₛ gₛ tₛ cₛ cₛ ^{m}cₛ [symbol] -3′ |
| SEQ ID NO: 23 | SH20 | 5′- [symbol] gₛ aₛ tₛ aₛ gₛ gₛ tₛ cₛ cₛ [symbol] -3′ |

```
SEQ ID NO: 1
Homo sapiens ATXN3 mRNA
NM_004993
      1 gagaggggca gggggcggag ctggaggggg tggttcggcg tgggggccgt
          tggctccaga
    61 caaataaaca tggagtccat cttccacgag aaacaagaag gctcactttg
          tgctcaacat
  121 tgcctgaata acttattgca aggagaatat tttagccctg tggaattatc
          ctcaattgca
  181 catcagctgg atgaggagga gaggatgaga atggcagaag gaggagttac
          tagtgaagat
  241 tatcgcacgt ttttacagca gccttctgga aatatggatg acagtggttt
          tttctctatt
  301 caggttataa gcaatgcctt gaaagtttgg ggtttagaac taatcctgtt
          caacagtcca
  361 gagtatcaga ggctcaggat cgatcctata aatgaaagat catttatatg
          caattataag
  421 gaacactggt ttacagttag aaaattagga aaacagtggt ttaacttgaa
          ttctctcttg
  481 acgggtccag aattaatatc agatacatat cttgcacttt tcttggctca
          attacaacag
  541 gaaggttatt ctatatttgt cgttaagggt gatctgccag attgcgaagc
          tgaccaactc
  601 ctgcagatga ttagggtcca acagatgcat cgaccaaaac ttattggaga
          agaattagca
  661 caactaaaag agcaaagagt ccataaaaca gacctggaac gagtgttaga
          agcaaatgat
  721 ggctcaggaa tgttagacga agatgaggag gatttgcaga gggctctggc
          actaagtcgc
  781 caagaaattg acatggaaga tgaggaagca gatctccgca gggctattca
          gctaagtatg
  841 caaggtagtt ccagaaacat atctcaagat atgacacaga catcaggtac
          aaatcttact
  901 tcagaagagc ttcggaagag acgagaagcc tactttgaaa aacagcagca
          aaagcagcaa
  961 cagcagcagc agcagcagca gcagggggac ctatcaggac agagttcaca
          tccatgtgaa
1021 aggccagcca ccagttcagg agcacttggg agtgatctag gtgatgctat
          gagtgaagaa
1081 gacatgcttc aggcagctgt gaccatgtct ttagaaactg tcagaaatga
          tttgaaaaca
1141 gaaggaaaaa aataatacct ttaaaaaata atttagatat tcatactttc
          caacattatc
1201 ctgtgtgatt acagcatagg gtccactttg gtaatgtgtc aaagagatga
          ggaaataaga
1261 cttttagcgg tttgcaaaca aaatgatggg aaagtggaac aatgcgtcgg
          ttgtaggact
1321 aaataatgat cttccaaata ttagccaaag aggcattcag caattaaaga
          catttaaaat
1381 agttttctaa atgtttcttt ttcttttttg agtgtgcaat atgtaacatg
          tctaaagtta
1441 gggcattttt cttggatctt tttgcagact agctaattag ctctcgcctc
          aggctttttc
1501 catatagttt gttttctttt tctgtcttgt aggtaagttg gctcacatca
          tgtaatagtg
1561 gctttcattt cttattaacc aaattaacct ttcaggaaag tatctctact
          ttcctgatgt
1621 tgataatagt aatggttcta gaaggatgaa cagttctccc ttcaactgta
          taccgtgtgc
1681 tccagtgttt tcttgtgttg ttttctctga tcacaacttt tctgctacct
          ggttttcatt
1741 attttcccac aattcttttg aaagatggta atcttttctg aggtttagcg
          ttttaagccc
1801 tacgatggga tcattatttc atgactggtg cgttcctaaa ctctgaaatc
          agccttgcac
1861 aagtacttga gaataaatga gcatttttta aaatgtgtga gcatgtgctt
          tcccagatgc
1921 tttatgaatg tcttttcact tatatcaaaa ccttacagct ttgttgcaac
          cccttcttcc
1981 tgcgccttat tttttccttt cttctccaat tgagaaaact aggagaagca
          tagtatgcag
2041 gcaagtctcc ttctgttaga agactaaaca tacgtaccca ccatgaatgt
          atgatacatg
2101 aaatttggcc ttcaatttta atagcagttt tattttattt tttctcctat
          gactggagct
2161 ttgtgttctc tttacagttg agtcatggaa tgtaggtgtc tgcttcacat
          cttttagtag
2221 gtatagcttg tcaaagatgg tgatctggaa catgaaaata atttactaat
          gaaaatatgt
2281 ttaaatttat actgtgattt gacacttgca tcatgtttag atagcttaag
          aacaatggaa
2341 gtcacagtac ttagtggatc tataaataag aaagtccata gttttgataa
          atattctctt
2401 taattgagat gtacagagag tttcttgctg ggtcaatagg atagtatcat
          tttggtgaaa
2461 accatgtctc tgaaattgat gttttagttt cagtgttccc tatccctcat
          tctccatctc
2521 cttttgaagc tcttttgaat gttgaattgt tcataagcta aaatccaaga
          aatttcagct
2581 gacaacttcg aaaattataa tatggtatat tgccctcctg gtgtgtggct
          gcacacattt
2641 tatcagggaa agttttttga tctaggattt attgctaact aactgaaaag
          agaagaaaaa
2701 atatctttta tttatgatta taaaatagct ttttcttcga tataacagat
          tttttaagtc
2761 attattttgt gccaatcagt tttctgaagt ttcccttaca caaaaggata
          gctttatttt
2821 aaaatctaaa gtttctttta atagttaaaa atgtttcaga aaaattataa
          aactttaaaa
2881 ctgcaaggga tgttggagtt tagtactact ccctcaagat ttaaaaagct
          aaatatttta
2941 agactgaaca tttatgttaa ttattaccag tgtgtttgtc atattttcca
          tggatatttg
3001 ttcattacct ttttccattg aaaagttaca ttaaactttt catacacttg
          aattgatgag
3061 ctacctaata taaaaatgag aaaaccaata tgcattttaa agttttaact
          ttagagttta
3121 taaagttcat atatacccta gttaaagcac ttaagaaaat atggcatgtt
          tgacttttag
3181 ttcctagaga gtttttgttt ttgtttttgt ttttttttga gacggagtct
          tgctatgtct
3241 cccaggctgg agggcagtgg catgatctcg gctcactaca acttccacct
          cccgggttca
3301 agcaattctc ctgcctcagc ctccagagta gctgggatta caggcgccca
          ccaccacacc
3361 cggcagattt ttgtattttt ggtagagacg cggtttcatc atgtttggcc
          aggctggtct
3421 cgaactcctg acctcaggtg atccgcctgc cttggcctcc caaagtgttg
          ggattacagg
3481 catgagccac tgcgcctggc cagctagaga gtttttaaag cagagctgag
          cacacactgg
3541 atgcgtttga atgtgtttgt gtagtttgtt gtgaaattgt tacatttagc
          aggcagatcc
3601 agaagcacta gtgaactgtc atcttggtgg ggttggctta aatttaattg
          actgtttaga
3661 ttccatttct taattgattg gccagtatga aaagatgcca gtgcaagtaa
          ccatagtatc
3721 aaaaaagtta aaaattattc aaagctatag tttatacatc aggtactgcc
          atttactgta
3781 aaccacctgc aagaaagtca ggaacaacta aattcacaag aactgtcctg
          ctaagaagtg
3841 tattaaagat ttccattttg ttttactaat tgggaacatc ttaatgttta
          atatttaaac
3901 tattggtatc atttttctaa tgtataattt gtattactgg gatcaagtat
          gtacagtggt
3961 gatgctagta gaagtttaag ccttggaaat accactttca tattttcaga
          tgtcatggat
4021 ttaatgagta atttatgttt ttaaaattca gaatagttaa tctctgatct
          aaaaccatca
4081 atttatgttt tttacggtaa tcatgtaaat atttcagtaa tataaactgt
          ttgaaaaggc
4141 tgctgcaggt aaactctata ctaggatctt ggccaaataa tttacaattc
          acagaatatt
4201 ttatttaagg tggtgctttt tttttttgtc cttaaaactt gatttttctt
          aactttattc
4261 atgatgccaa agtaaatgag gaaaaaaact caaaaccagt tgagtatcat
          tgcagacaaa
4321 actaccagta gtccatattg tttaatatta agttgaataa aataaatttt
          atttcagtca
4381 gagcctaaat cacattttga ttgtctgaat ttttgatact atttttaaaa
          tcatgctagt
4441 ggcggctggg cgtggtagct cacgcctgta atcccagcat tttgggaggc
          cgaagtgggt
4501 ggatcacgag gtcgggagtt cgagaccagc ttggccaaaa tggtgaaacc
          ccatctgtac
4561 taaaaactac aaaaattagc tgggcgcggt ggcaggtgcc tgtaatccca
          gctacctggg
4621 agtctgaggc aggagaattg cttgaaccct ggcgacagag gatgcagtga
          gccaagatgg
4681 tgccactgta ctccagactg ggcgacagag tgagactctg tctcaaaaaa
          aaaaaaaaaa
4741 tcatgctagt gccaagagct actaaattct taaaaccggc ccattggacc
          tgtacagata
4801 aaaaatagat tcagtgcata atcaaaatat gataatttta aaatcttaag
          tagaaaaata
4861 aatcttgatg ttttaaattc ttacgaggat tcaatagtta atattgatga
          tctcccggct
4921 gggtgcagtg gctcacgcct gtaatcccag cagttctgga ggctgaggtg
          ggcgaatcac
4981 ttcaggccag gagttcaaga ccagtctggg caacatggtg aaacctcgtt
          tctactaaaa
5041 atacaaaaat tagccgggcg tggttgcaca cacttgtaat cccagctact
          caggaggcta
5101 agaatcgcat gagcctagga ggcagaggtt gcagagtgcc aagggctcac
          cactgcattc
5161 cagcctgccc aacagagtga gacactgttt ctgaaaaaaa aaaatatata
          tatatatata
5221 tatatgtgtg tatatatata tgtatatata tatgacttcc tattaaaaac
          tttatcccag
5281 tcgggggcag tggctcacgc ctgtaatccc aacactttgg gaggctgagg
          caggtggatc
5341 acctgaagtc cggagtttga gaccagcctg gccaacatgg tgaaacccca
          tctctactaa
5401 aaatacaaaa cttaagccag gtatggtggc gggcacctgt aatcccagtt
          acttgggagg
5461 ctgaggcagg agaatcgttt aaacccagga ggtggaggtt gcagtgagct
          gagatcgtgc
5521 cattgcactc tagcctgggc aacaagagta aaactccatc ttaaaggttt
          gtttgttttt
5581 ttttaatccg gaaacgaaga ggcgttgggc cgctattttc tttttctttc
          tttctttctt
5641 tctttttttt tttttctgag acggagtcta gctctgctgc ccaggctgga
          gtacaatgac
5701 acgatgttgg ctcactgcaa cctccacctc ctgggttcaa gcgattctcc
          tgcctcagcc
5761 tcccaagtac ctgggattac aggcacctgc cactacacct ggcgaatatt
          tctttttttt
5821 agtagagacg ggcttttacc atgttaggct ggtctcaaac tcctgacctc
          aggtgatctg
5881 cctgccttgg cctcccaaag tgctgggatt acaggtgcag gccaccacac
          ccggccttgg
5941 gccactgttt tcaaagtgaa ttgtttgttg tatcgagtcc ttaagtatgg
          atatatatgt
6001 gaccctaatt aagaactacc agattggatc aactaatcat gtcagcaatg
          taaataactt
6061 tatttttcat attcaaaata aaaactttct tttatttctg gcccctttat
          aaccagcatc
6121 tttttgcttt aaaaaatgac ctggctttgt atttttttag tcttaaacat
          aataaaaata
6181 tttttgttct aatttgcttt catgagtgaa gattattgac atcgttggta
          aattctagaa
6241 ttttgatttt gttttttaat ttgaagaaaa tctttgctat tattattttt
          tccaagtggt
6301 ctggcatttt aagaattagt gctaataacg taacttctaa atttgtcgta
          attggcatgt
6361 ttaatagcat atcaaaaaac attttaagcc tgtggattca tagacaaagc
          aatgagaaac
6421 attagtaaaa tataaatgga tattcctgat gcatttagga agctctcaat
          tgtctcttgc
6481 atagttcaag gaatgttttc tgaatttttt taatgctttt tttttttttg
          aaagaggaaa
6541 acatacattt ttaaatgtga ttatctaatt tttacaacac tgggctatta
          ggaataactt
6601 tttaaaaatt actgttctgt ataaatattt gaaattcaag tacagaaaat
          atctgaaaca
6661 aaaagcattg ttgtttggcc atgatacaag tgcactgtgg cagtgccgct
          tgctcaggac
6721 ccagccctgc agcccttctg tgtgtgctcc ctcgttaagt tcatttgctg
          ttattacaca
6781 cacaggcctt cctgtctggt cgttagaaaa gccgggcttc caaagcactg
          ttgaacacag
6841 gattctgttg ttagtgtgga tgttcaatga gttgtatttt aaatatcaaa
          gattattaaa
6901 taaagataat gtttgctttt cta
SEQ ID NO: 2
Nucleotides 961-1020 of Homo sapiens ATXN3 mRNA (WT; not comprising
G987C SNP)
  961 cagcagcagc agcagcagca gcagggggac ctatcaggac agagttcaca
          tccatgtgaa
SEQ ID NO: 3
Nucleotides 977-997 of Homo sapiens ATXN3 mRNA (WT; not comprising
G987C SNP)
  977 agcagcaggg ggacctatca g
SEQ ID NO: 4
SNP ID rs12895357
SEQ ID NO: 5
Nucleotides 961-1020 of mutant Homo sapiens ATXN3, mRNA (comprising
G987C SNP)
  961 cagcagcagc agcagcagca gcagcgggac ctatcaggac agagttcaca
          tccatgtgaa
SEQ ID NO: 6
Nucleotides 977-997 of mutant Homo sapiens ATXN3, mRNA (comprising
G9870 SNP)
  977 agcagcagcg ggacctatca g
SEQ ID NO: 7
5′ - ATAGGTCCCGCTGCT - 3′
SEQ ID NO: 8
5′ - TGATAGGTCCCGCTGC - 3′
SEQ ID NO: 9
5′ - CTGATAGGTCCCGCTG - 3′
SEQ ID NO: 10
5′ - CTGATAGGTCCCGCT - 3′
SEQ ID NO: 11
5′ - CTGATAGGTCCCGC - 3′
SEQ ID NO: 12
5′ - ATAGGTCCCGC - 3′
SEQ ID NO: 13
mRNA Target for SH06
5′ - AGCAGCGGGACCUAU - 3′
SEQ ID NO. 14
mRNA Target for SH10
5′ - GCAGCGGGACCUAUCA - 3′
SEQ ID NO: 15
mRNA Target for SH13
5′ - CAGCGGGACCUAUCAG - 3′
SEQ ID NO: 16
mRNA Target for SH16
5′ - AGCGGGACCUAUCAG - 3′
SEQ ID NO: 17
mRNA Target for SH20
5′ - GCGGGACCUAUCAG - 3′
SEQ ID NO: 18
mRNA Target
5′ - GCGGGACCUAU - 3′
SEQ ID NO: 19
SH06
5′- [symbol]   gₛ gₛ tₛ cₛ cₛ ^{m}cₛ gₛ cₛ tₛ [symbol]   -3′
SEQ ID NO: 20
SH10
5′- [symbol]  tₛ aₛ gₛ gₛ tₛ cₛ cₛ ^{m}cₛ gₛ cₛ G C -3′
SEQ ID NO: 21
SH13
5′- [symbol]   aₛ tₛ aₛ gₛ gₛ tₛ cₛ cₛ ^{m}cₛ gₛ [symbol]   -3′
SEQ ID NO: 22
SH16
5′- [symbol]   aₛ tₛ aₛ gₛ gₛ tₛ cₛ cₛ ^{m}cₛ [symbol]  -3′
SEQ ID NO: 23
SH20
5′- [symbol]   gₛ aₛ tₛ aₛ gₛ gₛ tₛ cₛ cₛ [symbol]   -3,
```

1.-148. (canceled)

149. A single stranded oligonucleotide 8 to 30 nucleotides in length, wherein the oligonucleotide comprises a nucleotide sequence that is at least 90% identical to the reverse complement of a region of SEQ ID NO: 5 that includes position 25.

150. The oligonucleotide of claim 149, wherein the contiguous nucleotide sequence comprises no more than one mismatch with the reverse complement of SEQ ID NO: 5, and wherein the mismatch is not at position 25.

151. The oligonucleotide of claim 149, wherein the oligonucleotide is 12-18 nucleotides in length

152. The oligonucleotide of claim 149, comprising one or more sugar modified nucleotide analogues.

153. The oligonucleotide of claim 152, wherein the sugar modified nucleotide analogues are selected from the group consisting of locked nucleic acid (LNA), 2′-O-alkyl-RNA, 2′-OMe-RNA, 2′-amino-DNA and 2′-fluoro-DNA.

154. The oligonucleotide of claim 152, wherein the one or more nucleotide analogues are LNA.

155. The oligonucleotide of claim 152, wherein the one or more nucleotide analogues are oxy-LNA.

156. The oligonucleotide of claim 153, wherein the one or more nucleotide analogues are beta-D-oxy-LNA.

157. The oligonucleotide of claim 150, wherein the oligonucleotide comprise a sequence selected from the group consisting of: SEQ ID NO: 7, SEQ ID NO: 8, SEQ ID NO: 9, SEQ ID NO: 10, SEQ ID NO:11 and SEQ ID NO: 12.

158. The oligonucleotide according to claim 151, wherein the oligonucleotide is a gapmer.

159. The oligonucleotide of claim 149, wherein the oligonucleotide is selected from the group consisting of SEQ ID NO: 23, SEQ ID NO: 19, SEQ ID NO: 20, SEQ ID NO: 21 and SEQ ID NO: 22.

160. The oligonucleotide of claim 149, wherein the oligonucleotide inhibits the expression of ATXN3 mRNA in a cell which is expressing ATXN3 mRNA, wherein the ATXN3 mRNA encodes a pathogenic poly-glutamine expansion.

161. A conjugate comprising the oligonucleotide of claim 149 and at least one non-nucleotide moiety covalently attached to the oligonucleotide.

162. A pharmaceutical composition comprising the oligonucleotide of claim 149 a pharmaceutically acceptable diluent, carrier or adjuvant.

163. The use of the oligonucleotide of claim 149 for the treatment of spinocerebellar ataxia 3.

164. A method of treating a subject affected by spinocerebellar ataxia 3, the method comprising the step of administering the oligonucleotide of claim 149 to the subject, such that one or more objective symptoms of the spinocerebellar ataxia 3 are improved.

165. The method of claim 164, wherein the objective symptoms are selected from the group consisting of reduced spasticity, increased muscle tone and improved gait.

166. A method of reducing the expression of aberrant ATXN3 in a cell expressing aberrant ATXN3, the method comprising a step of contacting the cell with the oligonucleotide of claim 149, such that the expression of aberrant ATXN3 is reduced.

167. The method of claim 164, wherein the oligonucleotide comprises a sequence selected from the group consisting of SEQ ID NO: 11, SEQ ID NO: 7, SEQ ID NO: 8, SEQ ID NO: 9, SEQ ID NO: 10, SEQ ID NO: 12, SEQ ID NO: 19, SEQ ID NO: 20, SEQ ID NO: 21, SEQ ID NO: 22 and SEQ ID NO: 23.

168. The method of claim 164, wherein the oligonucleotide is administered intrathecally.


SEQUENCE LISTING (23 sequences)

SEQ ID NO: 1 - DNA, 6923 bp, Homo sapiens
gagaggggcagggggcggagctggagggggtggttcggcgtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 2 - DNA, 60 bp, Artificial
  Focused region of WT ATXN3
cagcagcagcagcagcagcagcagggggacctatcaggacagagttcacatccatgtgaa

SEQ ID NO: 3 - DNA, 21 bp, Artificial
  Focused region of WT ATXN3
agcagcagggggacctatcag

SEQ ID NO: 4 - skipped

SEQ ID NO: 5 - DNA, 60 bp, Artificial
  Focused region of WT ATXN3
cagcagcagcagcagcagcagcagcgggacctatcaggacagagttcacatccatgtgaa

SEQ ID NO: 6 - DNA, 21 bp, Artificial
  Focused region of WT ATXN3
agcagcagcgggacctatcag

SEQ ID NO: 7 - DNA, 15 bp, Artificial
  Antisense oligonucleotide
ataggtcccgctgct

SEQ ID NO: 8 - DNA, 16 bp, Artificial
  Antisense oligonucleotide
tgataggtcccgctgc

SEQ ID NO: 9 - DNA, 16 bp, Artificial
  Antisense oligonucleotide
ctgataggtcccgctg

SEQ ID NO: 10 - DNA, 15 bp, Artificial
  Antisense oligonucleotide
ctgataggtcccgct

SEQ ID NO: 11 - DNA, 14 bp, Artificial
  Antisense oligonucleotide
ctgataggtcccgc

SEQ ID NO: 12 - DNA, 11 bp, Artificial
  Antisense oligonucleotide
ataggtcccgc

SEQ ID NO: 13 - RNA, 15 nt, Artificial
  mRNA Target Sequence
agcagcgggaccuau

SEQ ID NO: 14 - RNA, 16 nt, Artificial
  mRNA Target Sequence
gcagcgggaccuauca

SEQ ID NO: 15 - RNA, 16 nt, Artificial
  mRNA Target Sequence
cagcgggaccuaucag

SEQ ID NO: 16 - RNA, 15 nt, Artificial
  mRNA Target Sequence
agcgggaccuaucag

SEQ ID NO: 17 - RNA, 14 nt, Artificial
  mRNA Target Sequence
gcgggaccuaucag

SEQ ID NO: 18 - RNA, 11 nt, Artificial
  mRNA Target Sequence
gcgggaccuau

SEQ ID NO: 19 - DNA, 15 bp, Artificial
  LNA oligonucleotide
ataggtcccgctgct

SEQ ID NO: 20 - DNA, 16 bp, Artificial
  LNA oligonucleotide
tgataggtcccgctgc

SEQ ID NO: 21 - DNA, 16 bp, Artificial
  LNA oligonucleotide
ctgataggtcccgctg

SEQ ID NO: 22 - DNA, 15 bp, Artificial
  LNA oligonucleotide
ctgataggtcccgct

SEQ ID NO: 23 - DNA, 14 bp, Artificial
  LNA oligonucleotie
ctgataggtcccgc